(19) United States
(12) Patent Application Publication
(10) Pub. No.: US 2025/0222009 A1
(43) Pub. Date: Jul. 10, 2025

(54) DINUCLEAR GOLD(I) COMPLEXES FOR TREATING CANCER

(71) Applicant: KING FAHD UNIVERSITY OF PETROLEUM AND MINERALS, Dhahran (SA)

(72) Inventors: Adam Ahmed Abdallah SULAIMAN, Dhahran (SA); Anvarhussien A. ISAB, Dhahran (SA); Abdul Malik Puthan PEEDIKAKKAL, Dhahran (SA)

(73) Assignee: KING FAHD UNIVERSITY OF PETROLEUM AND MINERALS, Dhahran (SA)

(21) Appl. No.: 18/406,937

(22) Filed: Jan. 8, 2024

(51) Int. Cl. A61K 31/555; A61P 35/00; C07F 1/00

(52) U.S. Cl. CPC A61K 31/555; A61P 35/00; C07F 1/00

(57) ABSTRACT

A dinuclear gold(I) complex has two gold(I) metal ion centers, two carbene ligands preferably having the same structure, and a diphosphane ligand comprising two phosphine groups. The two gold metal ions are coordinated to the two phosphine groups of the diphosphine ligand in a linear geometry. Further, each carbene ligand is independently connected to a gold metal ion centers via a carbon atom. The dinuclear gold(I) complex has an enhanced anticancer activity compared to an equimolar amount of cisplatin administered to a subject under substantially the same conditions.

### STATEMENT REGARDING PRIOR DISCLOSURE BY THE INVENTORS

Aspects of this technology are described in N. Casagrande, C. Borghese, G. Corona, D. Aldinucci, M. Altaf, A. Sulaiman, A. Isab, S. Ahmad, and A. Peedikakkal “Dinuclear gold(I) complexes based on carbene and diphosphane ligands: bis[2-(dicyclohexylphosphano)ethyl]amine complex inhibits the proteasome activity, decreases stem cell markers and spheroid viability in lung cancer cells” published in Journal of Biological Inorganic Chemistry, Volume 28, 751-766, which is incorporated herein by reference in its entirety.

### STATEMENT OF ACKNOWLEDGEMENT

This research was supported by the Interdisciplinary Research Center for Advanced Materials under the project No. INAM2210, at King Fahd University of Petroleum & Minerals, Dhahran, Saudi Arabia; and the Italian Association for Cancer Research (D.A.) and Italian Ministry of Health (Ricerca Corrente).

### BACKGROUND

### Technical Field

The present disclosure is directed to gold(I) complexes, and more particularly relates to dinuclear phosphane gold(I) complexes having carbene and diphosphane ligands and methods of treating cancer.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

The potential of gold(I) complexes as anticancer agents has opened a new horizon for scientific research on metal-based therapeutic agents. Gold(I) complexes containing N-heterocyclic carbenes (NHCs) and phosphanes exhibit anticancer activity with no cross-resistance with cisplatin. These complexes generally possess a linear structure. The NHCs are efficient σ-donors and are, therefore, form strong bonds to the metal center. The ligand exchange reactions of various [(NHC)AuX]complexes indicate a strong trans effect of the NHC and a preferred exchange of X as the leaving group.

Gold complexes may interact with proteins of the redox control systems and/or with the proteasome machinery, suggesting that their anticancer activity may originate at these two levels.

In particular, gold(I) complexes can bind to the selenocysteine residue present in the C-terminal active site of the enzyme thioredoxin reductase (TrxR). TrxR is a target for the cytotoxic activity of gold compounds. It scavenges oxidants and free radicals and protects against oxidative stress damage. Another target for various gold complexes is the 20S proteasome. As the proteasome represents the central component of the protein degradation machinery, namely the Ubiquitin-Proteasome-System (UPS), its inhibition by gold(I) complexes may affect the maintenance of protein homeostasis and inhibit cell survival.

The cytotoxicity of gold(I)-carbene complexes containing an IPr moiety, such as [Au(IPr)₂]BF₄, is effective due to the lipophilic character and the enhanced promotion of mitochondrial perturbation, causing cancer cell death through the induction of the apoptotic pathway. A series of heteroleptic IPr-based bis carbene gold(I) complexes that displayed higher anti-cancer potency in lung cancer cells (A549) with respect to gold(I) auranofin have been described (See: Sen, S.; Li, Y.; Lynch, V.; Arumugam, K; Sessler, J. L.; Arambula, J. F. Expanding the Biological Utility of Bis-NHC Gold(I) Complexes through Post Synthetic Carbamate Conjugation. Chem. Commun. 2019, 55, 10627-10630). More recently, the synthesis and cytotoxicity of 7 gold(I) complexes based on IPr and different phosphane ligands were found to make the complexes more lipophilic by increasing their cellular uptake (See: Sulaiman, A. A. A.; Casagrande, N.; Borghese, C.; Corona, G.; Isab, A. A.; Ahmad, S.; Aldinucci, D.; Altaf M. Design, Synthesis, and Preclinical Activity in Ovarian Cancer Models of New Phosphanegold(I)—N-Heterocyclic Carbene Complexes. J Med Chem 2022, 65, 14424-14440). These gold(I) complexes had high toxicity in ovarian cancer cells (OvCa) with no cross-resistance with cisplatin. These gold(I) complexes also exhibit higher activity than the reference drug cisplatin in 2D and 3D models (multicellular spheroids) of OvCa.

Lung cancer has a high mortality rate worldwide because it is an incurable malignancy resistant to conventional therapy. Despite the discovery of specific molecular targets and new treatment strategies for lung cancer, there is a need to develop more efficient therapies, especially to eliminate cancer stem cells (CSCs). CSCs are subpopulations of cancer cells within tumors with self-renewal capabilities, which maintain and generate new tumors, are more prone to form metastasis, evade tumor immunity, and are resistant to many anti-cancer drugs. Thus, eliminating, or at least reducing, their number is a challenge in cancer treatment.

Although a few gold(I) complexes have been developed in the past, there still remains a need to develop complexes with enhanced lipophilicity and increased cellular uptake for cancer treatment.

In view of the foregoing, one objective of the present disclosure is to provide a dinuclear gold(I) complex for tumor inhibition. The dinuclear gold(I) complex may contain carbene, e.g., 1,3-Bis(2,6-di-isopropylphenyl)imidazol-2-ylidene (IPr)) and diphosphane ligands, such as [bis(1,2-diphenylphosphano)ethane (Dppe), bis(1,3-diphenylphosphano)propane (Dppp) and bis[2-(dicyclohexylphosphano)ethyl]amine (DCyPA)]. A second objective of the present disclosure is to provide a pharmaceutical composition that contains a dinuclear gold(I) complex.

A third objective of the present disclosure is to provide a method for inhibiting proliferation of cancer cells. A fourth objective of the present disclosure is to provide a method of treating lung cancer in a subject.

### SUMMARY

In an exemplary embodiment, a dinuclear gold(I) complex is described. The complex includes two gold(I) metal ion centers, two carbene ligands having the same structure, and a diphosphane ligand comprising two phosphine groups. In some embodiments, the two gold metal ions are coordinated to the two phosphine groups of the diphosphine ligand in a linear geometry. In some embodiments, each carbene ligand is independently connected to a gold metal ion centers via a carbon atom. In some embodiments, the dinuclear gold(I) complex has an enhanced anticancer activity compared to an equimolar amount of cisplatin administered to a subject under substantially the same conditions.

In some embodiments, the dinuclear gold(I) complex has a formula (I)

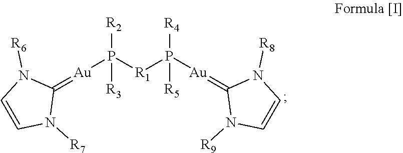

In some embodiments, R₂, R₃, R₄, R₅, R₆, R₇, R₈ and R₉ are each independently selected from the group consisting of unsubstituted or substituted 5, 6, 7 or 8-membered rings.

In some embodiments, R₁ is independently C₁-C₆ alkyl, hydroxy-C₁-C₆ alkyl, C₁-C₆ alkyloxy, C₁-C₆ alkylthio, C₁-C₆ alkenyl, C₂-C₆ alkynyl, C₁-C₆ alkyloxy-C₁-C₆ alkyl, C₁-C₆ alkylthio-C₁-C₆ alkyl, C₁-C₆ alkylamino, di-(C₁-C₆ alkyl) amino, and C₁-C₆ alkylamino-C₁-C₆ alkyl.

In some embodiments, each of the carbon-containing radicals is optionally substituted with one or more halogen atoms.

In some embodiments, R₆, R₇, R₈ and R₉ are each independently phenyl that is substituted in the 2 and 6 positions with at least one of hydrogen, alkyl, or aryl.

In some embodiments, R₆, R₇, R₈ and R₉ are phenyl substituted at the 2 and 6 positions with isopropyl.

In some embodiments, R₂, R₃, R₄, and R₅ are each independently selected from the group consisting of unsubstituted phenyl and unsubstituted cyclohexyl.

In some embodiments, R₁ is independently selected from the group consisting of C₁-C₆ alkyl, and C₁-C₆ alkylamino-C₁-C₆ alkyl.

In some embodiments, R₁ is independently selected from the group consisting of —C₂H₄—, —C₃H₆—, and —(C₂H₄)₂N—.

In some embodiments, the diphosphane ligand is bis(1,2-diphenylphosphano)ethane (Dppe), and the complex has a formula (II)

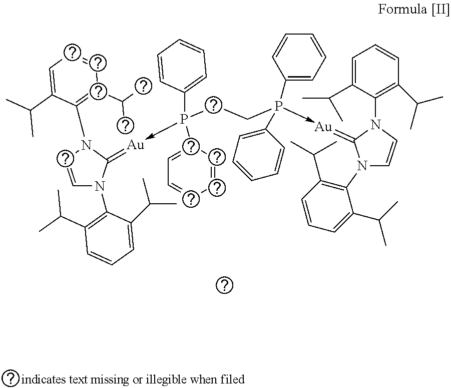

In some embodiments, the dinuclear gold(I) complex of formula (II) exhibits a cancer inhibition activity with a half maximal inhibitory concentration (IC₅₀) of 0.16 to 0.40 micromolar (M).

In some embodiments, the diphosphane ligand is bis(1,3-diphenylphosphano)propane (Dppp), and the complex has a formula (III)

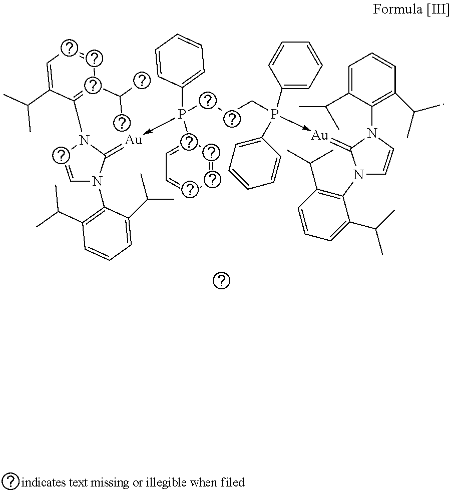

In some embodiments, the dinuclear gold(I) complex of formula (III) exhibits a cancer inhibition activity with IC₅₀ of 0.2 to 0.50 μM.

In some embodiments, the diphosphane ligand is bis[2-(dicyclohexylphosphano)ethyl]amine (DCyPA), and the complex has a formula (IV)

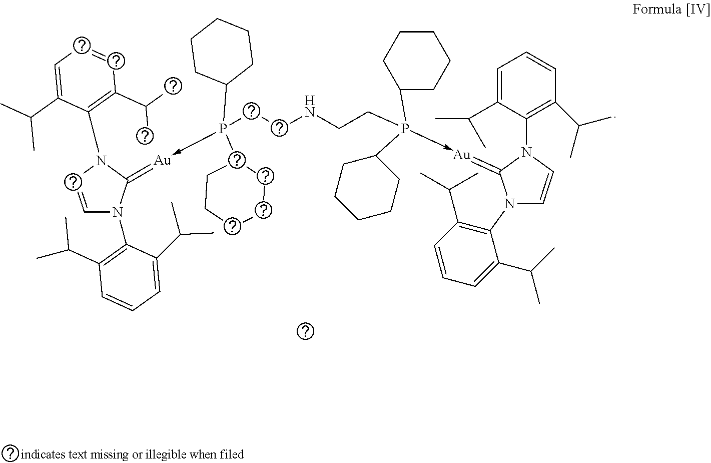

In some embodiments, the dinuclear gold(I) complex of formula (IV) exhibits a cancer inhibition activity with IC₅₀ of 0.1 to 0.50 μM.

In an exemplary embodiment, a pharmaceutical composition is described. The pharmaceutical complex includes at least one dinuclear gold(I) complex, at least one pharmaceutically acceptable carrier or excipient and, and optionally, at least one other anticancer drug, chemotherapeutic agent, or immunopotentiator.

In an exemplary embodiment, a method for inhibiting proliferation of cancer cells is described. The method includes contacting the cancer cells with a cytotoxic effective amount of the dinuclear gold(I) complex.

In some embodiments, the cancer cells comprise one or more cancer stem cells selected from the group consisting of a breast cancer stem, a lung cancer stem cell, a prostate cancer stem cell, an osteosarcoma cancer stem cell, an ovarian cancer stem cell, a colon carcinoma stem cell, and a melanoma stem cell.

In some embodiments, the cytotoxic effective amount of the dinuclear gold(I) complex is from 0.01 to 5 μM.

In some embodiments, the dinuclear gold(I) complex exhibits an IC₅₀ of from 0.1 to 0.5 μM for inhibiting the proliferation and inducing the apoptosis of lung cancer cells.

In some embodiments, the lung cancer cells are A549 lung cancer stem cells.

In an exemplary embodiment, a method of treating lung cancer in a subject, is described. The method includes administering to the subject the dinuclear gold(I) complex in an amount effective to decrease the average cancer cell viability of the lung cancer by more than 10%.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 shows the chemical structure and resonance assignment of binuclear gold(I) complexes 1, 2, and 3, according to certain embodiments;

FIG. 2 shows high-resolution mass spectra of the binuclear gold(I) complexes 1, 2, and 3 (designated as C1, C2, and C3, respectively), according to certain embodiments;

FIG. 3 shows a molecular structure view of C2, according to certain embodiments;

FIG. 4 shows a molecular structure view of C3, according to certain embodiments;

FIG. 5A shows effect of C3 on the cell viability in A549 cells, as observed by crystal violet staining, according to certain embodiments;

FIG. 5B is a flow cytometric analysis chart depicting the effect of C3 on apoptosis (Annexin-V-FITC plus 7AAD) in A549 cells, according to certain embodiments;

FIG. 5C shows representative flow cytometry dot plot of Annexin-V-FITC/7AAD stained cells, according to certain embodiments;

FIG. 5D shows the effect of C3 on apoptosis by caspase-3,7 activation, as evaluated by flow cytometry with fluorochrome-labeled inhibitors of caspases (FLICA) after 24 h incubation, according to certain embodiments;

FIG. 5E shows the effect of C3 on BAX and Bcl-2 expression, as evaluated after 72 h treatment with C3 by flow cytometry, according to certain embodiments;

FIG. 5F shows the effect of C3 on mitochondrial reactive oxygen species (mitROS) generation, after 48 h incubation, according to certain embodiments;

FIG. 5G shows representative flow cytometry histograms of reactive oxygen species (ROS) after treatment with C3, according to certain embodiments;

FIG. 5H is a histogram showing the percentage of cells in each cell cycle phase evaluated after propidium iodide staining and flow cytometry analysis in A549 cells after treatment for 24 h with C3, according to certain embodiments;

FIG. 5I shows representative flow cytometry histograms of the cell cycle progression after drug treatment, according to certain embodiments;

FIG. 6 shows the effect of C1 and C2 on the cell viability in A549 cells, as observed by crystal violet staining, according to certain embodiments;

FIG. 7A shows the effect of C3 on 20S-proteasome, according to certain embodiments;

FIG. 7B shows the effect of C3 on NF-kB p65 transcription factor, according to certain embodiments;

FIG. 7C shows the effect of C3 on thioredoxin reductase (TrxR) activity, according to certain embodiments;

FIG. 8 shows the effect of C3 on cancer stem cell markers (ALDH1, CD133, CD44 and NOTCH1), according to certain embodiments;

FIG. 9A shows the effect of C3 on viability of 3D-A549-MCTSs, according to certain embodiments;

FIG. 9B shows the effect of cisplatin on the viability of 3D-A549-MCTSs, according to certain embodiments;

FIG. 9C shows representative phase contrast micro-photographs showing the cytotoxic effects of C3 and cisplatin in multiple size A549-MCTSs, according to certain embodiments;

FIG. 10 shows ¹H NMR spectrum of complex 1 in CDCl₃, according to certain embodiments;

FIG. 11 shows ¹³C NMR spectrum of complex 1 in DMSO-d₆, according to certain embodiments;

FIG. 12 shows ³¹P NMR spectrum of complex 1 in DMSO-d₆, according to certain embodiments;

FIG. 13 shows ¹H NMR spectrum of complex 2 in DMSO-d₆, according to certain embodiments;

FIG. 14 shows ¹³C NMR spectrum of complex 2 in CDCl₃+DMSO-d₆, according to certain embodiments;

FIG. 15 shows ³¹P NMR spectrum of complex 2 in DMSO-d₆, according to certain embodiments;

FIG. 16 shows ¹H NMR spectrum of complex 3 in CDCl₃, according to certain embodiments;

FIG. 17 shows ¹³C NMR spectrum of complex 3 in CDCl₃, according to certain embodiments; and

FIG. 18 shows ³¹P NMR spectrum of complex 3 in CDCl₃, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

As used herein, the words “about,” “approximately,” or “substantially similar” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), or +/−20% of the stated value (or range of values). Within the description of this disclosure, where a numerical limit or range is stated, the endpoints are included unless stated otherwise. Also, all values and subranges within a numerical limit or range are specifically included as if explicitly written out.

As used herein, the term “cancer” refers to all types of cancer, neoplasm, or malignant tumors found in mammals (e.g., humans), including but not limited to leukemias, lymphomas, carcinomas, and sarcomas. Exemplary cancers that may be treated with the method or composition provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with the method provided herein include cancer of the thyroid, endocrine system, brain, breast, cervix, colon, head and neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulinoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the term “half maximal inhibitory concentration (IC₅₀)” refers to the measure of the potency of a substance in inhibiting a specific biological or biochemical function. IC₅₀ is a quantitative measure that shows how much of a particular inhibitory substance (e.g., drug) is needed to inhibit, in vitro, a given biological process or biological component by 50%. The biological component can be an enzyme, cell, cell receptor, or microorganism. In some embodiments, IC₅₀ values are expressed as molar concentration, preferably, e.g., moles per liter.

As used herein, “analogue” refers to a chemical compound that is structurally similar to a parent compound, but differs slightly in composition (e.g., one atom or functional group is different, added, or removed). The analogue may or may not have chemical or physical properties different from the original compound and may or may not have improved biological and/or chemical activity. For example, the analogue may be more hydrophilic, or it may have altered reactivity as compared to the parent compound. The analogue may mimic the chemical and/or biologically active of the parent compound (i.e., it may have similar or identical activity), or, in some cases, may have increased or decreased activity. The analogue may be a naturally or non-naturally occurring variant of the original compound. Other types of analogues include isomers (enantiomers, diastereomers, and the like) and other types of chiral variants of a compound, as well as structural isomers.

As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have chemical or physical properties different from the parent compound. For example, the derivative may be more hydrophilic, or it may have altered reactivity as compared to the parent compound. Derivatization (i.e., modification) may involve the substitution of one or more moieties within the molecule (e.g., a change in a functional group). The term “derivative” also includes conjugates, and prodrugs of a parent compound (i.e., chemically modified derivatives that can be converted into the original compound under physiological conditions).

The term “therapeutically effective amount” as used herein refers to the amount of the complex being administered, which will relieve to some extent one or more of the symptoms of the disorder being treated. In reference to cancer or pathologies related to increased cell division, a therapeutically effective amount refers to that amount which has the effect of at least one of the following: (1) reducing the size of a tumor, (2) inhibiting (that is, slowing to some extent, preferably stopping) aberrant cell division, growth or proliferation, for example, cancer cell division, (3) preventing or reducing the metastasis of cancer cells, (4) relieving to some extent (or, preferably, eliminating) one or more symptoms associated with a pathology related to or caused in part by unregulated or aberrant cellular division, including for example, cancer and (5) inducing apoptosis of cancer cells or tumor cells.

As used herein, the terms “therapies” and “therapy” can refer to any method(s), composition(s), and/or agent(s), and/or complexes that can be used in the prevention, treatment and/or management of cancer or one or more symptoms thereof.

As used herein, the terms “treat,” “treatment,” and “treating” in the context of the administration of a therapy to a subject in need thereof refer to the reduction or inhibition of the progression and or duration of cancer, the reduction or amelioration of the severity of cancer, and/or the amelioration of one or more symptoms thereof resulting from the administration of one or more therapies. In some embodiments, the subject is a mammalian subject. In one embodiment, the subject is a human. The term “treating” or “treatment” of a disease includes preventing the disease from occurring in a subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer or hyperplasia, these terms simply mean that the life expectancy of an individual affected with cancer will be increased or that one or more of the symptoms of the disease will be reduced. In specific embodiments, such terms refer to one, two or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%, and (12) an increase in the number of patients in remission. In certain embodiments, such terms refer to a stabilization or reduction in cancer stem cell population. In some embodiments, such terms refer to a stabilization or reduction in the growth of cancer cells. In some embodiments, such terms refer to stabilization or reduction in cancer stem cell population and a reduction in the cancer cell population. In some embodiments, such terms refer to a stabilization or reduction in the growth and or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in the survival rate of a patient population. In further embodiments, such terms refer to an increase in the response rate, the durability of response, or the number of patients who respond or are in remission. In some embodiments, such terms refer to a decrease in the hospitalization rate of a patient population and/or a decrease in hospitalization length for a patient population.

A “pharmaceutical composition” refers to a mixture of the compounds described herein or pharmaceutically acceptable salts, esters, or prodrugs thereof, with other chemical components, such as physiologically acceptable carriers and excipients. In the present disclosure, a pharmaceutical composition may be used to facilitate the administration of at least one gold(I) complex to a subject.

The terms “pharmaceutically acceptable salt” and “pharmaceutically acceptable ester” generally refer to a compound in a pharmaceutically acceptable form such as an ester, a phosphate ester, a salt of an ester, or a related) which, upon administration to a subject in need thereof, provides at least one of the gold(I) complexes deserved herein. Pharmaceutically acceptable salts and esters retain the biological effectiveness and properties of the free bases, which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like. Suitable salts include those derived from alkali metals such as potassium and sodium, and alkaline earth metals such as calcium and magnesium, among numerous other acids well-known in the art.

As used herein, a “pharmaceutically acceptable carrier” generally refers to a carrier or diluent that does not cause significant irritation to an organism and does not abrogate the biological activity and properties of the administered gold(I) complex. The term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005, which is incorporated herein by reference in its entirety. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

As used herein, the term “excipient” generally refers to an inert substance added to a pharmaceutical composition to facilitate the administration of a compound further. Examples, without limitation, of excipients include calcium carbonate, calcium phosphate, various sugars and types of starch, cellulose derivatives, gelatin, vegetable oils, and polyethylene glycols.

As used herein, the term “alkyl”, unless otherwise specified, generally refers to a straight, or branched hydrocarbon fragment. Non-limiting examples of such hydrocarbon fragments include methyl, ethyl, propyl, isopropyl, butyl, isobutyl, t-butyl, pentyl, isopentyl, neopentyl, hexyl, isohexyl, 3-methylpentyl, 2,2-dimethylbutyl, and 2,3-dimethylbutyl.

As used herein, the term “substituted” generally refers to at least one hydrogen atom that is replaced with a non-hydrogen group, provided that normal valencies are maintained and that the substitution results in a stable compound. When a compound or a R group (denoted as R₁, R₂, and so forth) is noted as “optionally substituted”, the substituents are selected from the exemplary group including, but not limited to, aroyl (as defined hereinafter), halogen (e.g. chlorine, bromine, fluorine or iodine), alkoxy (i.e. straight or branched chain alkoxy having 1 to 10 carbon atoms, and includes, for example, methoxy, ethoxy, propoxy, isopropoxy, butoxy, isobutoxy, secondary butoxy, tertiary butoxy, pentoxy, isopentoxy, hexyloxy, heptyloxy, octyloxy, nonyloxy, and decyloxy), cycloalkyloxy including cyclopentyloxy, cyclohexyloxy, and cycloheptyloxy, aryloxy including phenoxy and phenoxy substituted with halo, alkyl, alkoxy, and haloalkyl (which refers to straight or branched chain alkyl having 1 to 8 carbon atoms which are substituted by at least one halogen, and includes, for example, chloromethyl, bromomethyl, fluoromethyl, iodomethyl, 2-chloroethyl, 2-bromoethyl, 2-fluoroethyl, 3-chloropropyl, 3-bromopropyl, 3-fluoropropyl, 4-chlorobutyl, 4-fluorobutyl, dichloromethyl, dibromomethyl, difluoromethyl, diiodomethyl, 2,2-dichloroethyl, 2,2-dibromoethyl, 2,2-difluoroethyl, 3,3-dichloropropyl, 3,3-difluoropropyl, 4,4-dichlorobutyl, 4,4-difluorobutyl, trichloromethyl, trifluoromethyl, 2,2,2-tri-fluoroethyl, 2,3,3-trifluoropropyl, 1,1,2,2-tetrafluoroethyl, 2,2,3,3-tetrafluoropropyl), hydrocarbyl, arylalkyl, hydroxy, alkoxy, oxo, alkanoyl, alkanoyloxy, amino, alkylamino, arylamino, arylalkylamino, disubstituted amines (e.g. in which the two amino substituents are selected from the exemplary group including, but not limited to, alkyl, aryl, or arylalkyl), alkanylamino, arylamino, alkanoylamino, thiol, alkylthio, arylthio, arylalkylthio, alkylthiono, arylthiono, aryalkylthiono, alkylsulfonyl, arylsulfonyl, arylalkylsulfonyl, sulfonamido (e.g. SO₂NH₂), substituted sulfonamide, nitro, cyano, carboxy, carbamyl (e.g. CONH₂, CONHalkyl, CONHaryl, CONHarylalkyl or cases where there are two substituents on one nitrogen from alkyl, aryl, or arylalkyl), alkoxycarbonyl, aryl, guanidine, heteroarylcarbonyl, heterocyclyl, and mixtures thereof and the like. The substituents may be either unprotected, or protected as necessary, as known to those skilled in the art, for example, as taught in Greene, et al., “Protective Groups in Organic Synthesis”, John Wiley and Sons, Second Edition, 1991, hereby incorporated by reference in its entirety).

As used herein, the term “unsubstituted alkyl” generally refers to an alkyl group which may be linear or branched and does not have any hydrogen atom that is replaced with a non-hydrogen group. Exemplary unsubstituted alkyl group includes, without limitation, methyl, ethyl, propyl, isopropyl, butyl, tert-butyl, sec-butyl, isobutyl, pentyl, and hexyl.

As used herein, the term “alkylthio” as used in this disclosure generally refers to a divalent sulfur with alkyl occupying one of the valencies and includes the groups methylthio, ethylthio, propylthio, butylthio, pentylthio, hexylthio, and octylthio.

The present disclosure may further include all isotopes of atoms occurring in the present compounds. Isotopes include those atoms having the same atomic number but different mass numbers. By way of general example, and without limitation, isotopes of hydrogen include deuterium and tritium. Isotopes of carbon include ¹³C and ¹⁴C. Isotopically labeled compounds of the disclosure can generally be prepared by conventional techniques known to those skilled in the art or by processes and methods analogous to those described herein, using an appropriate isotopically-labeled reagent in place of the non-labeled reagent otherwise employed.

Aspects of this disclosure are directed to dinuclear gold(I) complexes (also referred to as a complex) containing carbene, preferably, e.g., (1,3-bis(2,6-di-isopropylphenyl)imidazol-2-ylidene (IPr)), and diphosphane ligands, preferably, e.g., [bis(1,2-diphenylphosphano)ethane (Dppe), bis(1,3-diphenylphosphano)propane (Dppp) and bis[2-(dicyclohexylphosphano)ethyl]amine (DCyPA)]. The described complexes were characterized by various analytical techniques and further tested for its anti-cancer potential in lung (A549), breast (MC-F7), prostate (PC-3), osteosarcoma (MG-63), and ovarian (A2780 and A2780cis) cancer models. The results show that the growth inhibition of the complexes of the present disclosure is higher than cisplatin in all cell lines tested.

A dinuclear gold(I) complex is described. The gold complex is dinuclear with two gold(I) metal ion centers or precursors thereof. Suitable examples of gold precursors include compounds selected from, but are not limited to, chloro(triethylphosphine)-gold(I), chloro(trimethylphosphine)gold(I), chloro[diphenyl(o-tolyl)phosphine]gold(I), chloro[tri(o-tolyl)phosphine]gold(I), chloro(methyldiphenylphosphine)gold(I), chloro[2-(dicyclohexyl phosphino)-biphenyl]gold(I), chloro[2-di-tert-butyl(2′,4′,6′-triisopropylbiphenyl)phosphine]gold(I), chloro[di(1-adamantyl)-2-dimethylaminophenylphosphine]gold(I), chloro(2-dicyclo hexyl-phosphino-2′-dimethylaminobiphenyl)gold(I), chloro(trimethyl phosphite)gold(I), chloro[(1,1′-biphenyl-2-yl)di-tert-butylphosphine]gold(I), chloro[2-dicyclohexyl(2′,4′,6′-trisopropyl-biphenyl)phosphine]gold(I), chloro[tris(2,3,4,5,6-pentafluorophenyl)-phosphine]gold(I), chloro[tri(p-tolyl)phosphine]gold(I), chloro[2-dicyclohexyl(2′,6′-dimethoxybiphenyl)-phosphine]gold(I), chloro[2-dicyclohexyl(2′,6′;-diisopropoxybiphenyl)-phosphine]gold(I), chloro[2-dicyclohexylphosphino-2′,6′-bis(N,N-dimethylamino)-biphenyl]gold(I), chloro {4-[2-di(1-adamantyl)phosphino]phenylmorpholine}gold(I), chloro(2-di-tert-butylphosphino-3,6-dimethoxy-2′,4′,6′-triisopropylbiphenyl)gold(I), chloro[2-(dicyclohexylphosphino)-3,6-dimethoxy-2′,4′,6′-triisopropyl-1,1′-biphenyl]gold(I), chloro(2-{bis[3,5-bis(trifluoromethyl)-phenyl]phosphino}-3,6-dimethoxy-2′,4′,6′-triisopropyl-1,1′-biphenyl)gold(I), and chloro(2-di-tert-butylphosphino-3,4,5,6-tetramethyl-2′,4′,6′-triisopropylbiphenyl)gold(I).

The complex further includes two carbene ligands, and a diphosphane ligand comprising two phosphine groups. Each gold atom is bonded to both the ligands—namely the carbene ligand, and the diphosphane ligand. In some embodiments, each of the two carbene ligands is independently connected to each of the two gold metal ion centers via a carbon atom of the corresponding carbene ligand. In some embodiments, the two carbene ligands may have the same or a different structure. In a preferred embodiment, the two carbene ligands have the same structure. In some embodiments, the carbene ligand is an imidazol-2-ylidene-based compound or derivatives thereof. In a specific embodiment, the carbene ligand is (1,3-bis(2,6-di-isopropylphenyl)imidazol-2-ylidene (IPr).

In some embodiments, the two gold (I) metal ions are further connected to/coordinated with each other via each of the two phosphine atoms of the diphosphine ligand. In some embodiments, the diphosphine ligand includes two phosphorous atoms—a first phosphorous atom bonded to a first gold (I) metal ion and a second phosphorous atom bonded to a second gold (I) metal ion. In some embodiments, the diphosphine ligand serves as a binding bridge that binds the two gold (I) metal ions in a linear geometry. Preferred examples of the diphosphane ligand may include [bis(1,2-diphenylphosphano)ethane (Dppe), bis(1,3-diphenylphosphano)propane (Dppp) and bis[2-(dicyclohexylphosphano)ethyl]amine (DCyPA)]. In some embodiments, the complex of the present disclosure shows an enhanced anticancer activity compared to cisplatin. In a preferred embodiment, the anticancer activity of the complex in the present disclosure is at least 10%, at least 20%, at least 40%, at least 80, or at least 150% higher than that of the cisplatin, as determined using methods described herein or known to those of skill in the art, including but not limited to cell viability assays (MTT and CCK-8), clonogenic assays, apoptosis assays (flow cytometry and Annexin V/PI staining), DNA damage and repair assays (Comet assay and γ-H2AX staining), cell cycle analysis, and in vivo tumor models.

In an embodiment, the complex is a compound of formula (I)

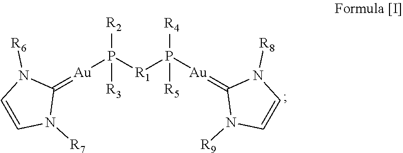

In some embodiments, R₁ is independently C₁-C₆ alkyl, hydroxy-C₁-C₆ alkyl, C₁-C₆ alkyloxy, C₁-C₆ alkylthio, C₁-C₆ alkenyl, C₂-C₆ alkynyl, C₁-C₆ alkyloxy-C₁-C₆ alkyl, C₁-C₆ alkylthio-C₁-C₆ alkyl, C₁-C₆ alkylamino, di-(C₁-C₆ alkyl) amino, and C₁-C₆ alkylamino-C₁-C₆ alkyl. In some further embodiments, each of the carbon-containing radicals is optionally substituted with one or more halogen atoms. In some preferred embodiments, R₂, R₃, R₄, R₅, R₆, R₇, R₈, and R₉ are each independently selected from the group consisting of unsubstituted or substituted 5, 6, 7, or 8-membered rings.

In some embodiments, R₆, R₇, R₈ and R₉ are each independently phenyl that is substituted in the 2 and 6 positions with at least one of hydrogen, alkyl, or aryl. In a specific embodiment, R₆, R₇, R₈, and R₉ are each independently phenyl that is substituted in the 2 and 6 positions with isopropyl. In some embodiments, R₂, R₃, R₄, and R₅ are each independently selected from the group consisting of unsubstituted phenyl and unsubstituted cyclohexyl.

In some embodiments, R₁ is independently selected from the group consisting of C₁-C₆ alkyl, and C₁-C₆ alkylamino-C₁-C₆ alkyl. In a specific embodiment, R₁ is independently selected from the group consisting of —C₂H₄—, —C₃H₆—, and —(C₂H₄)₂N—.

In some embodiments, the diphosphane ligand is bis(1,2-diphenylphosphano)ethane (Dppe), and is compound (1) of Formula [II]

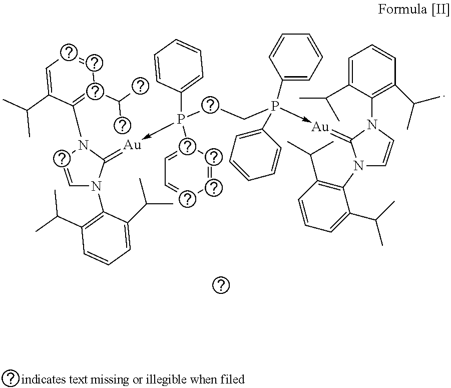

In some embodiments, the compound (1) of Formula [II] exhibits a cancer inhibition activity with a half maximal inhibitory concentration (IC₅₀) of 0.16 to 0.40 micromolar (μM), preferably 0.18 to 0.38 μM, preferably 0.20 to 0.36 μM, preferably 0.22 to 0.34 μM, preferably 0.24 to 0.32 μM, preferably 0.26 to 0.30 μM, or even more preferably about 0.28 μM, as determined by an in vitro cytotoxicity method against one or more cancer cell lines selected from the group consisting of A549, non-small cell lung cancer cell, PC3, androgen-resistant prostate cancer, MCF-7, breast cancer, and MG-63, osteosarcoma. Other ranges are also possible.

In some embodiments, the diphosphane ligand is bis(1,3-diphenylphosphano)propane (Dppp), and is compound (2) of formula [III]

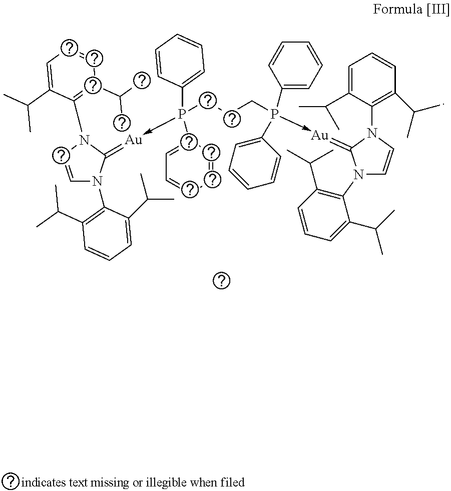

In some embodiments, the compound (2) of Formula [III] exhibits a cancer inhibition activity with a IC₅₀ of 0.2 to 0.50 μM, preferably 0.24 to 0.46 μM, preferably 0.28 to 0.42 μM, preferably 0.32 to 0.38 μM, or even more preferably about 0.36 μM, as determined by the in vitro cytotoxicity method against one or more cancer cell lines selected from the group consisting of A549, non-small cell lung cancer cell, PC3, androgen-resistant prostate cancer, MCF-7, breast cancer, and MG-63, osteosarcoma. Other ranges are also possible.

In some embodiments, the diphosphane ligand is bis[2-(dicyclohexylphosphano) ethyl]amine (DCyPA), and is a compound (3) of Formula [IV].

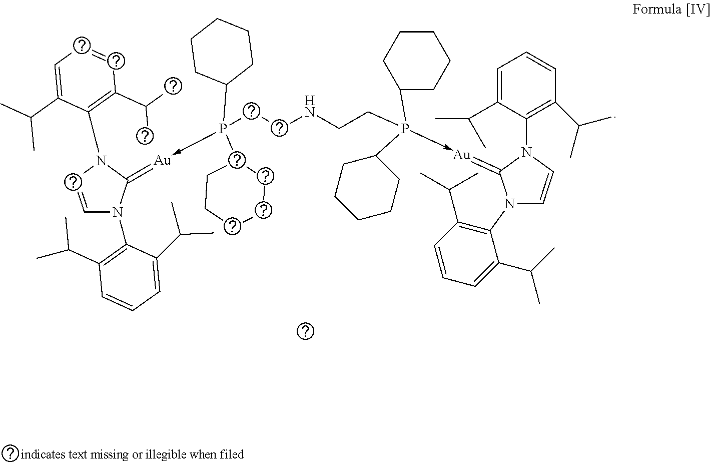

In some embodiments, the compound (3) of Formula [IV] exhibits a cancer inhibition activity with IC₅₀ of 0.1 to 0.50 μM, preferably 0.14 to 0.46 μM, preferably 0.18 to 0.42 μM, preferably 0.22 to 0.38 μM, preferably 0.26 to 0.34 μM, preferably 0.30 to 0.34 μM, or even more preferably about 0.32 μM, as determined by the in vitro cytotoxicity method against one or more cancer cell lines selected from the group consisting of A549, non-small cell lung cancer cell, PC3, androgen-resistant prostate cancer, MCF-7, breast cancer, and MG-63, osteosarcoma. Other ranges are also possible.

The structures of the compounds (1-3) of formula (II-IV) may be characterized by Fourier transforms infrared spectroscopy (FT-IR). In some embodiments, the FT-IR may be collected in a Perkin Elmer FT-IR 180 spectrophotometer acquired in a range of 4000 to 400 centimeter inverse (cm⁻¹) at 4 cm⁻¹ resolution. 20 scans were carried out for each sample.

The structures of the compounds (1-3) of formula (II-IV) may be characterized by nuclear magnetic resonance (NMR) spectroscopy. In some embodiments, the NMR may be collected in a JEOL-LA 500 NMR spectrophotometer.

¹H and ¹³C NMR spectra may be recorded using the residual DMSO-d₆ at δ 2.50 ppm, ¹³C DMSO-d₆ signal at δ 39.52 ppm, CDCl₃-d at δ 7.24 ppm, and ¹³C CDCl₃-d signal at δ 77.23 ppm, as internal standards. The ³¹P NMR chemical shifts were recorded relative to external reference (H₃PO₄ in D₂O) at 0.00 ppm.

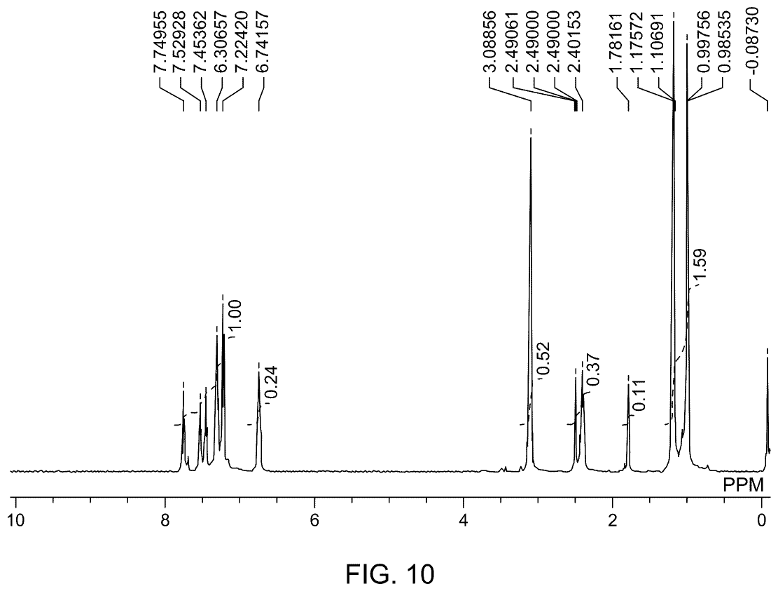

Referring to FIG. 10, ¹H NMR spectra of C1 of formula (II) in CDCl₃. In some embodiments, the C1 of formula (II) has peaks in a range of 7.7 to 7.85, preferably about 7.75; 7.4 to 7.6, preferably 7.47 to 7.53; 7.2 to 7.4, preferably about 7.22 to 7.31; 6.6 to 6.8, preferably about 6.74; 2.3 to 2.5, preferably about 2.40; 1.7 to 1.85, preferably about 1.78; 1.05 to 1.25, preferably about 1.14; 0.9 to 1.05, preferably about 0.99, as depicted in FIG. 10. Other ranges are also possible.

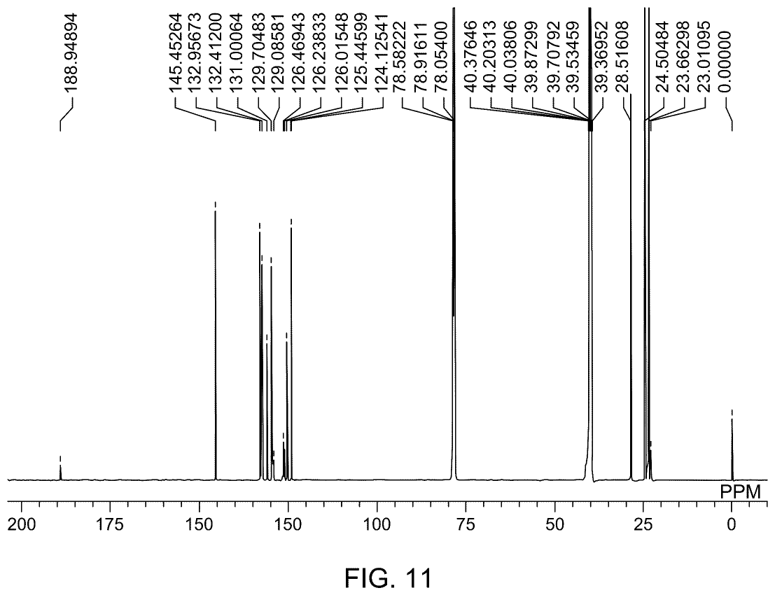

Referring to FIG. 11, ¹³C NMR spectra of C1 of formula (II) in CDCl₃. In some embodiments, the C1 of formula (II) in CDCl₃ has peaks in a range of about 145.4, about 131.0, about 133.0, about 124.1, about 28.5, about 23.7, about 24.5, about 124.1, about 188.9, about 23.0, about 126.3, about 132.4, about 129.7, and about 125.4, as depicted in FIG. 11. Other ranges are also possible.

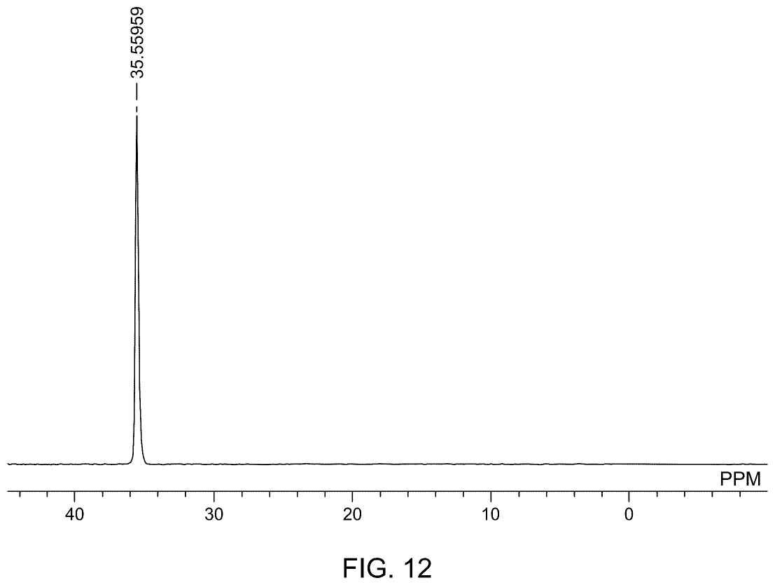

Referring to FIG. 12, ³¹P NMR spectra of C1 of formula (II) in CDCl₃. In some embodiments, the C1 of formula (II) in CDCl₃ has peaks in a range of 30 to 40, preferably about 35.56, as depicted in FIG. 12. Other ranges are also possible.

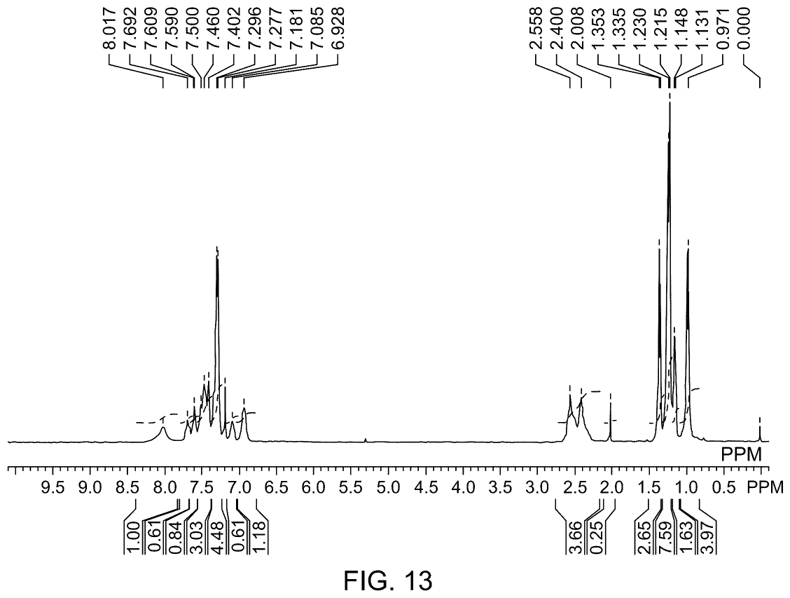

Referring to FIG. 13, ¹H NMR spectra of C2 of formula (III) in CDCl₃. In some embodiments, the C2 of formula (III) has peaks in a range of about 77.65, about 7.40, about 2.40, about 1.22, about 0.97, about 8.02, about 1.34, about 1.14, about 6.93, about 7.69, about 7.50, and about 7.18, as depicted in FIG. 13. Other ranges are also possible.

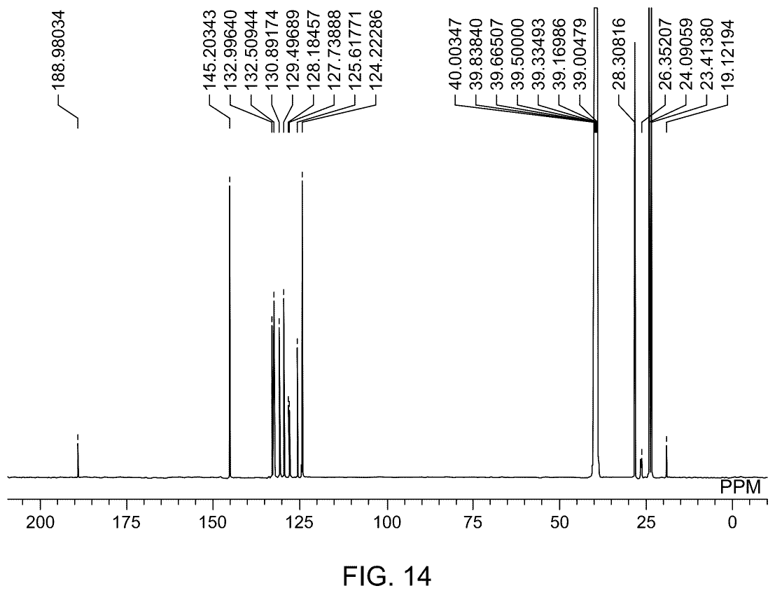

Referring to FIG. 14, ¹³C NMR spectra of C2 of formula (III) in CDCl₃. In some embodiments, the C2 of formula (III) in CDCl₃ has peaks in a range of about 145.2, about 130.9, about 133.0, about 124.2, about 28.3, about 24.1, about 23.4, about 124.2, about 189.0, about 26.4, about 19.1, about 128.0, about 132.5, about 129.5, and about 125.6, as depicted in FIG. 14. Other ranges are also possible.

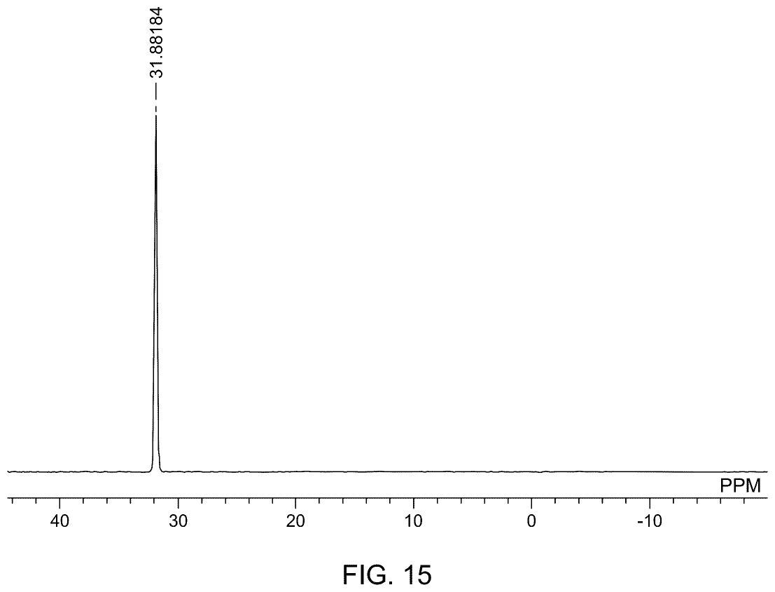

Referring to FIG. 15, ³¹P NMR spectra of C2 of formula (III) in CDCl₃. In some embodiments, the C2 of formula (III) in CDCl₃ has peaks in a range of 30 to 40, preferably about 31.88, as depicted in FIG. 15. Other ranges are also possible.

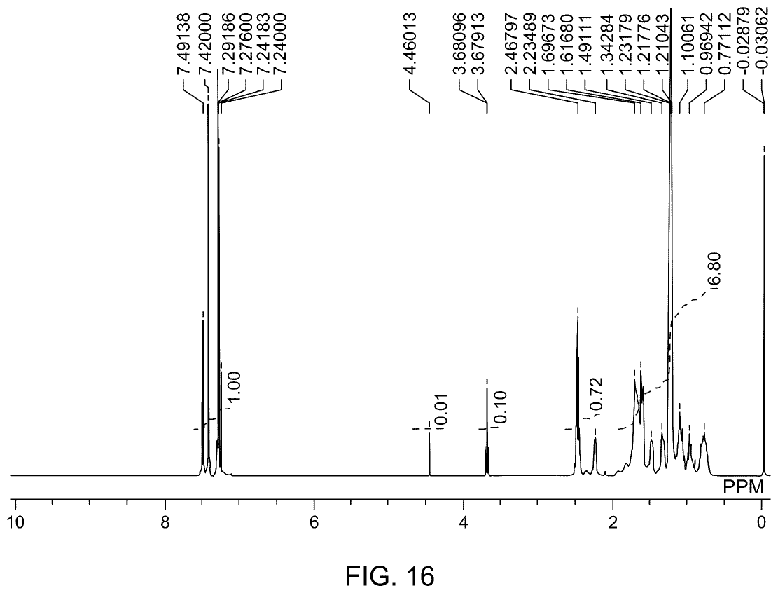

Referring to FIG. 16, ¹H NMR spectra of C3 of formula (IV) in CDCl₃/DMSO-d₆. In some embodiments, the C3 of formula (IV) has peaks in a range of about 7.42, about 7.24, about 2.47, about 1.23, about 1.21, about 7.49, about 1.70, about 2.23, about 1.62, about 1.49, about 1.43, and about 1.10, as depicted in FIG. 16. Other ranges are also possible.

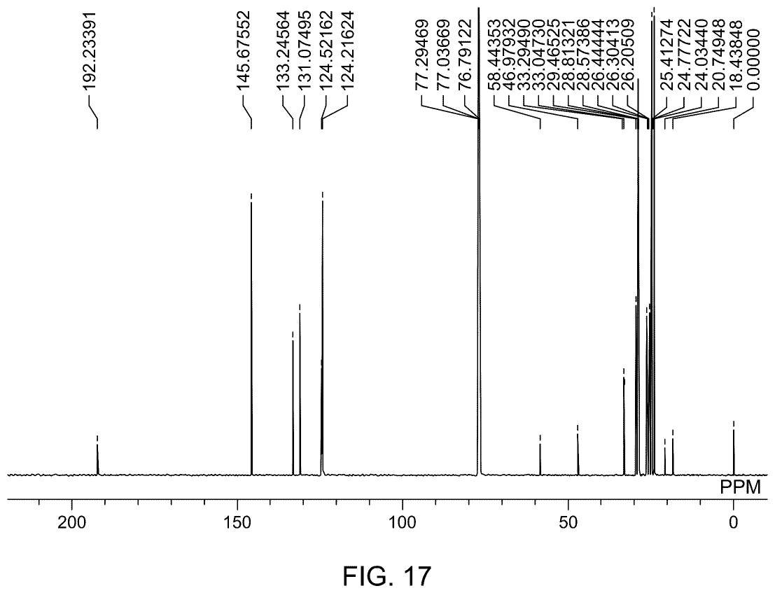

Referring to FIG. 17, ¹³C NMR spectra of C3 of formula (IV) in CDCl₃/DMSO-d₆. In some embodiments, the C3 of formula (IV) in CDCl₃/DMSO-d₆ has peaks in a range of about 145.7, about 131.0, about 133.2, about 124.5, about 28.8, about 24.0, about 24.8, about 124.2, about 192.2, about 33.2, about 47.0, about 26.4, about 29.5, about 28.6, and about 25.4, as depicted in FIG. 17. Other ranges are also possible.

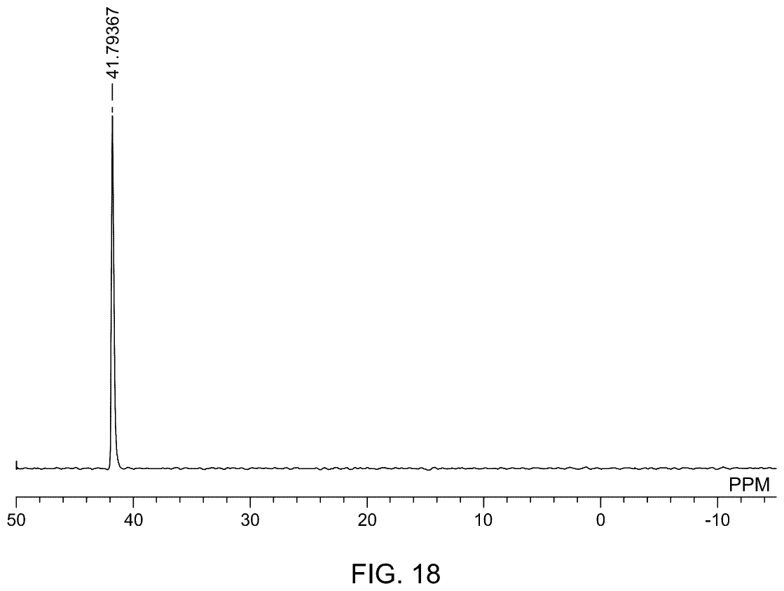

Referring to FIG. 18, ³¹P NMR spectra of C3 of formula (IV) in CDCl₃. In some embodiments, the C3 of formula (IV) in CDCl₃ has peaks in a range of 35 to 45, preferably about 41.79, as depicted in FIG. 18. Other ranges are also possible.

Another aspect of the present disclosure relates to a pharmaceutical composition comprising one or more of the complexes described herein. In other words, the complex described herein, or analogues or derivatives thereof, can be provided in a pharmaceutical composition. Depending on the intended mode of administration, the pharmaceutical composition can be in the form of solid, semi-solid, or liquid dosage forms, such as, for example, tablets, suppositories, pills, capsules, powders, liquids, or suspensions, preferably in a unit dosage form suitable for single administration of a precise dosage. The compositions will include a therapeutically effective amount of one or more of the gold complexes described herein or derivatives thereof in combination with a pharmaceutically acceptable carrier and, in addition, may include other medicinal agents, pharmaceutical agents, carriers, diluents, excipients, and optionally, one or more anticancer drug (s), chemotherapeutic agent (s), or immunopotentiator (s). By pharmaceutically acceptable, it meant a material that is not biologically or otherwise undesirable, which can be administered to an individual along with the selected compound without causing significant unacceptable biological effects or interacting in a deleterious manner with the other components of the pharmaceutical composition in which it is contained.

The neoplastic activity of the tumor or cancer cells may be localized or initiated in one or more of the following: blood, brain, bladder, lung, cervix, ovary, colon, rectum, pancreas, skin, prostate gland, stomach, breast, liver, spleen, kidney, head, neck, testicle, bone (including bone marrow), thyroid gland, central nervous system.

A pharmaceutical composition including the complex of the present disclosure can then be administered orally, systemically, parenterally, by inhalation spray, rectally, or topically in dosage unit formulations containing conventional nontoxic pharmaceutically acceptable carriers, adjuvants, and vehicles as desired. In some embodiments, the method of administration of the steroid or an analogue or derivative thereof is oral. In other embodiments, the compound or an analogue or derivative thereof is administered by injection, such as, for example, through a peritumoral injection.

Topical administration can also involve the use of transdermal administration, such as transdermal patches or iontophoresis devices. The term parenteral, as used herein, includes intravesical, intradermal, transdermal, subcutaneous, intramuscular, intralesional, intracranial, intrapulmonary, intracardial, intrasternal, and sublingual injections, or infusion techniques. Formulation of drugs is discussed in, for example, Hoover, John E., Remington's Pharmaceutical Sciences, Mack Publishing Co., Easton, Pa.; 1975. Another example includes Liberman, H. A. and Lachman, L., Eds., Pharmaceutical Dosage Forms, Marcel Decker, New York, N.Y., 1980, which is incorporated herein by reference in its entirety.

Injectable preparations, for example, sterile injectable aqueous or oleaginous suspensions, can be formulated according to the known art using suitable dispersing or wetting agents and suspending agents. The sterile injectable preparation can also be a sterile injectable solution or suspension in a nontoxic, parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, any fixed oil can be employed, including synthetic mono- or diglycerides. In addition, fatty acids such as oleic acid find use in the preparation of injectables. Dimethyl acetamide, surfactants including ionic and non-ionic detergents, and polyethylene glycols can be used. Mixtures of solvents and wetting agents, such as those discussed above, are also useful. Suppositories for rectal administration of the compound or an analogue or derivative thereof can be prepared by mixing the steroid or an analogue or derivative thereof with a suitable non-irritating excipient such as cocoa butter, synthetic mono- di- or triglycerides, fatty acids, and polyethylene glycols that are solid at ordinary temperatures but liquid at the rectal temperature and will therefore melt in the rectum and release the drug.

Solid dosage forms for oral administration can include capsules, tablets, pills, powders, and granules. In such solid dosage forms, the compounds of this disclosure are ordinarily combined with one or more adjuvants appropriate to the indicated route of administration. If administered by mouth, a contemplated steroid or an analogue or derivative thereof can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in a dispersion of the active compound in hydroxypropyl methylcellulose. In the case of capsules, tablets, and pills, the dosage forms can also comprise buffering agents such as sodium citrate, magnesium or calcium carbonate, or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. A contemplated steroid or an analogue or derivative thereof of the present disclosure can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending, agents, and sweetening, flavouring, and perfuming agents. The amount of active ingredient that can be combined with the carrier materials to produce a single dosage form varies depending upon the mammalian subject treated and the particular mode of administration.

Cancers such as but not limited to sarcomas, carcinomas, melanomas, myelomas, gliomas, and lymphomas can be treated or prevented with the complex provided herein. In some embodiments, a pharmaceutical composition incorporating the complex of the present disclosure is present in an amount effective for treating a patient having a proliferative disorder selected from the group consisting of head and neck cancer, breast cancer, lung cancer, colon cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), testicular cancer, and the like. In some embodiments, the cancer is preferably one of breast cancer, adenocarcinoma, breast adenocarcinoma, colon cancer, colorectal cancer, lung cancer, prostate cancer, kidney cancer, liver cancer, melanoma, pancreatic cancer, renal cancer, hepatocellular cancer, cervical cancer, testicular cancer, and the like.

The methods for treating cancer and other proliferative disorders described herein inhibit, remove, eradicate, reduce, regress, diminish, arrest, or stabilize a cancerous tumor, including at least one of the tumor growth, tumor cell viability, tumor cell division, and proliferation, tumor metabolism, blood flow to the tumor and metastasis of the tumor.

In some embodiments, a method for inhibiting the proliferation of cancer cells is described. The method includes contacting the cancer cells with a cytotoxic effective amount of the dinuclear gold(I) complex of the present disclosure. The cytotoxic effective amount of the dinuclear gold(I) complex is from 0.01 to 5 μM, preferably 0.05 to 4.5 μM, preferably 0.1 to 0.4 μM, preferably 0.15 to 0.35 μM, preferably 0.2 to 0.3 μM, or even more preferably about 0.25 μM. Other ranges are also possible. The cancer cells include one or more cancer stem cells selected from the group consisting of a breast cancer stem, a lung cancer stem cell, a prostate cancer stem cell, an osteosarcoma cancer stem cell, an ovarian cancer stem cell, a colon carcinoma stem cell, and a melanoma stem cell. In some embodiments, the cancer cells include lung cancer cells. The lung cancer cells are A549 lung cancer stem cells. The dinuclear gold(I) complex exhibits an IC₅₀ of from 0.1 to 0.5 μM, preferably 0.05 to 4.5 μM, preferably 0.1 to 0.4 μM, preferably 0.15 to 0.35 μM, preferably 0.2 to 0.3 μM, or even more preferably about 0.25 μM for inhibiting the proliferation and inducing the apoptosis of lung cancer cells.

In some embodiments, after treatment with one or more gold complexes or a pharmaceutical composition thereof, the size of a tumor, whether by volume, weight, or diameter, is reduced by at least 5%, 10%, 15%, 20%, 25%, 30%, 40%, 50%, 60%, 70%, 75%, 80%, 85%, 90%, 95%, 99% or 100%, relative to the tumor size before treatment. In other embodiments, after treatment with one or more gold complexes of a pharmaceutical composition thereof, the size of a tumor does not reduce but is maintained the same as the tumor size before treatment. Methods of assessing tumor size include but are not limited to CT scan, MRI, DCE-MRI, and PET Scan.

In some embodiments, the method for treating cancer and other proliferative disorders involves the administration of a unit dosage or a therapeutically effective amount of the gold complexes or a pharmaceutical composition thereof to a mammalian subject (preferably a human subject) or a patient in need thereof. As used herein, “a subject in need thereof” refers to a mammalian subject, preferably a human subject, who has been diagnosed with, is suspected of having, is susceptible to, is genetically predisposed to, or is at risk of having at least one form of cancer. A method of treating lung cancer in a subject is described. The method includes administering to the subject the dinuclear gold(I) complex in an amount effective to decrease the average cancer cell viability of lung cancer by more than 10%, preferably more than 20%, preferably more than 40%, preferably more than 80%, or even more preferably more than 150% based on a total number of the cancer cell viability of lung cancer. Other ranges are also possible.

The dosage and treatment duration is dependent on factors such as the bioavailability of a drug, administration mode, toxicity of a drug, gender, age, lifestyle, body weight, the use of other drugs and dietary supplements, cancer stage, tolerance, and resistance of the body to the administered drug, etc., then determined and adjusted accordingly. One or more gold complexes or a pharmaceutical composition thereof may be administered in a single dose or multiple individual divided doses. In some embodiments, the interval of time between the administration of complex or a pharmaceutical composition thereof and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks. 15-20 weeks, 20-30 weeks, 30-40 weeks, 40-50 weeks, 1 month, 2 months, 3 months, 4 months, 5 months, 6 months, 7 months, 8 months, 9 months, 10 months, 11 months, 12 months, 1 year, 2 years, or any period in between. In certain embodiments, complex and one or more additional therapies are administered less than 1 day, 1 week, 2 weeks, 3 weeks, 4 weeks, one month, 2 months, 3 months, 6 months, 1 year, 2 years, or 5 years apart. Other ranges are also possible.

In certain embodiments, the complex of the present disclosure or a pharmaceutical composition thereof may be used in combination with one or more other antineoplastic or chemotherapeutic agents. A non-limiting list of examples of chemotherapeutic agents are aflibercept, asparaginase, bleomycin, busulfan, carmustine, chlorambucil, cladribine, cyclophosphamide, cytarabine, dacarbazine, daunorubicin, doxorubicin, etoposide, fludarabine, gemcitabine, hydroxyurea, idarubicin, ifosamide, irinotecan, lomustine, mechelorethamine, melphalan, mercaptopurine, methotrexate, mitomycin, mitoxantrone, pentostatin, procarbazine, 6-thioguanine, topotecan, vinblastine, vincristine, retinoic acid, oxaliplatin, cisplatin, carboplatin, 5-FU (5-fluorouracil), teniposide, amasacrine, docetaxel, paclitaxel, vinorelbine, bortezomib, clofarabine, capecitabine, actinomycin D, epirubicine, vindesine, methotrexate, tioguanine (6-thioguaniue), tipifarnib. Examples of antineoplastic agents which are protein kinase inhibitors include imatinib, erlotinib, sorafenib, sunitinib, dasatinib, nilotinib, lapatinib, gefitinib, temsirolimus, everolimus, rapamycin, bosutinib, pazopanib, axitinib, neratinib, vatalanib, pazopanib, midostaurin, and enzastaurin. Examples of antineoplastic agents which are antibodies comprise trastuzumab, cetuximab, panitumumab, rituximab, bevacizumab, mapatumumab, conatumumab, lexatumumab, and the like.

### Examples

The disclosure will now be illustrated with working examples, which are intended to demonstrate the working of disclosure and not intended to restrictively imply any limitations on the scope of the present disclosure.

### Example 1: Materials and Instrumentation

All solvents were of analytical grade and used without further purification. Ethanol, diethyl ether, dichloromethane, and acetone were obtained from Fluka AG (St. Gallen, Switzerland). Cholorido(1,3-bis(2,6-di-isopropylphenyl)imidazol-2-ylidene)gold(I), [Au(IPr)Cl] and diphosphanes [bis(1,2-diphenylphosphano)ethane (Dppe), bis(1,3-diphenyl phosphano) propane (Dppp) and bis[2-(dicyclohexylphosphano)ethyl]amine (DCyPA)] were obtained from Strem Chemicals Inc. (Newburyport, Massachusetts. United States). AgPF₆ was obtained from Sigma-Aldrich, United States. The ¹H, ¹³C, and ³¹P nuclear magnetic resonance (NMR) spectra were recorded on a JEOL-LA 500 NMR spectrophotometer (manufactured by JEOL, Japan), operating at 500.0, 125.65, and 202.0 MHz, respectively, corresponding to a magnetic field of 11.74 T. The spectral conditions for ¹³C NMR included 32 k data points, a 3.2 s acquisition time, and a 5.75 s pulse width. ¹³C NMR spectra were obtained with ¹H broadband decoupling and the following spectral conditions: 32 k data points, a 1 s acquisition time, a 2.5 s pulse delay, and a 5.12 s pulse width. All spectra were recorded at 297 K in CDCl₃ relative to tetramethylsilane (TMS) as an internal standard. The ³¹P NMR chemical shifts were recorded relative to an external reference (H₃PO₄ in D₂O) at 0.00 ppm. Elemental analysis was obtained on Perkin Elmer Series 11 (CHNS/O) and Analyzer 2400 (manufactured by Perkin Elmer, United States). The solid-state Fourier Transform Infrared (FT-IR) spectra of the free ligands and their gold(I) compounds were recorded on a Perkin Elmer FT-IR 180 spectrophotometer (manufactured by Perkin Elmer, United States), using KBr pellets over the range 4000-400 cm⁻¹ at a resolution of 4 cm⁻¹.

### Example 2: High-Resolution Mass Spectrometry Analysis

High-resolution electrospray ionization (ESI)-mass spectrometry (MS) analysis of the three (compound 1 to 3) gold(I) complexes was performed by Orbitrap Qexactive Plus Mass spectrometer (Thermo Scientific, Massachusetts, United States). Samples, dissolved in water/acetonitrile (preferably e.g., about 1:1) solution at concentrations of about preferably e.g., 1 μM, and preferably e.g., 10 μL, were flow injected through the ionization source (Heated-ESI, HESI) into the Orbitrap mass analyzer operated in positive ionization mode and flowed at about 0.1 ml/min: source voltage about 2.0 KV, capillary temperature about 320° C. and source heater temperature about 50° C. Data were acquired at resolution 70,000 with a Fourier transform (FT)-MS instrument in a range of mass-to-charge ratios (m/z) of about 700-900 a.m.u. with AGC 3×106 and maximum injection time about 100 ms.

### Example 3: Synthesis of the Complexes

The complexes (1-3) were synthesized by adding 0.127 g (0.5 mmol) AgPF₆ dissolved in 5 mL of ethanol to a solution of 0.311 g (0.5 mmol) [Au(IPr)Cl] in 5 mL dichloromethane. The mixture was stirred at ambient temperature for 15 minutes and filtered off. 0.25 mmol of the corresponding diphosphane, Dppe, Dppp, and DCyPA in 5 mL dichloromethane was added to the filtrate. The mixture was stirred for 30 minutes and then filtered. The filtrate was kept in an undisturbed area. After five days, the colorless solids were obtained. For the DCyPA complex (3), colorless crystals were obtained and subjected to single crystal diffraction analysis.

### Example 4: Characterization

The preparation of [(Au(IPr))₂(C₂₆H₂₄P₂)](PF₆)₂ (complex 1 or C1) has a yield about 0.308 g, (66%). Anal. Calc. for C₈OH₉₆Au₂N₄P₄F₁₂ (1859.45 g/mol): C, 51.67; H, 5.20; N, 3.01. Found: C, 51.42; H, 6.22; N, 3.32. IR (cm⁻¹) ν(═CH) 3172, ν(CH₂) 2963 asym, 2870 sym, ν(C—H) 1329 bend, ν(C═C) 1467, ν(C—N) 1216, ν(P—C) 1106. ¹H NMR (CDCl₃, ppm) δ 7.53 (H3), 7.31 (H4), 2.40 (H5), 1.14 (d, H6), 0.99 (d, H7), 7.75 (m, H8), 1.78 (H10), 6.74 (H12), 7.47 (H13). 7.22 (H14). ¹³C NMR (CDCl₃, ppm) δ 145.4 C(1), 131.0 C(2), 133.0 C(3), 124.1 C(4), 28.5 C(5), 23.7 C(6), 24.5 C(7), 124.1 C(8), 188.9 C(9), 23.0 C(10), 126.3 C(11), 132.4 C(12), 129.7 C(13), 125.4 C(14). ³¹P NMR (CDCl₃, ppm) δ 35.56.

The preparation of [(Au(IPr))₂(C₂₇H₂₆P₂)](PF₆)₂ (complex 2 or C2) has a yield about 0.332 g, (71%). Anal. Calc. for C₈₁H₉₈Au₂N₄P₄F₁₂ (1873.48 g/mol): C, 51.92; H, 5.27; N, 2.99. Found: C, 51.85; H, 5.04; N, 3.02. IR (cm⁻¹) ν(═CH) 3172, ν(CH2) 2962 asym, 2870 sym, ν(C—H) 1329 bend, ν(C═C) 1469, ν(C—N) 1215, ν(P—C) 1105. ¹H NMR (CDCl₃, ppm) δ 7.65 (H3), 7.40 (H4), 2.40 (H5), 1.22 (d, H6), 0.97 (d, H7), 8.02 (m, H8), 1.34 (H10), 1.14 (H11), 6.93 (H12), 7.69 (H13). 7.50 (H14), 7.18 (H15). ¹³C NMR (CDCl₃, ppm) δ 145.2 C(1), 130.9 C(2), 133.0 C(3), 124.2 C(4), 28.3 C(5), 24.1 C(6), 23.4 C(7), 124.2 C(8), 189.0 C(9), 26.4 C(10), 19.1 C(11), 128.0 C(12), 132.5 C(13), 129.5 C(14), 125.6 C(15). ³¹P NMR (CDCl₃, ppm) δ 31.88.

The preparation of [(Au(IPr))₂(C₂₈H₂₄P₂)](PF₆)₂ (complex 3 or C3) has a yield about 0.290 g (60%). Anal. calc. for C₈₂H₁₂₅Au₂N₅P₄F₁₂ (1926.71 g/mol): C, 51.11; H, 6.53; N, 3.63. Found: C, 50.68; H, 6.52; N, 3.45. IR (cm⁻¹) ν(N—H) 3333, ν(═CH) 3172, ν(CH₂) 2920 asym, 2852 sym, ν(C—H) 1329 bend, ν(C═C) 1469, ν(C—N) 1215, ν(P—C) 1117. ¹H NMR (CDCl₃/DMSO-d6, ppm) δ 7.42 (H3), 7.24 (H4), 2.47 (H5), 1.23 (d, H6), 1.21 (d, H7), 7.49 (m, H8), 1.70 (H10), 2.23 (H11), 1.62 (H12), 1.49 (H13), 1.43 (H14), 1.10 (H15). ¹³C NMR (CDCl₃/DMSO-d6, ppm) δ 145.7 C(1), 131.0 C(2), 133.2 C(3), 124.5 C(4), 28.8 C(5), 24.0 C(6), 24.8 C(7), 124.2 C(8), 192.2 C(9), 33.2 C(10), 47.0 C(11), 26.4 C(12), 29.5 C(13), 28.6 C(14), 25.4 C(15). ³¹P NMR (CDCl₃, ppm) δ 41.79.

### Example 5: X-Ray Structure Determination of Complex 2 and Complex 3

Suitable crystals of complex 1 were obtained as colorless blocks from dichloromethane/ethanol solution. Intensity data for C2 were collected on a Bruker AXS D8 Venture diffractometer using MoK_{α} (λ=0.71073 Å) radiation generated by I μs microfocus tube. Data were recorded with a Bruker AXS PHOTON II CPAD detector. The data collection was performed at about 304 K using ω- and φ-scans. The data was processed using APEX3 Crystal Structure Analysis Package (Bruker AXS). Data collection: APEX3, structure solution: direct methods (SHELXT-2014) (See: Sheldrick, G. M. A Short History of SHELX Acta Cryst A 2008, 64, 112-122, which is incorporated herein by reference in its entirety) and structure refinement: full-matrix least-squares method on F² (SHELXL-2014) (See: Stoe & Cie. (2009). X-Area & X-RED32. Stoe & Cie GmbH, Darmstadt, Germany, which is incorporated herein by reference in its entirety) using ShelXle as the graphical user interface. The crystal data and details of data collection and refinement of complex 2 are summarized in Table 1.

TABLE 1
Crystal data and details of the structure refinement for complexes 2 and 3.

| | Details | | | |
|---|---|---|---|---|
| Parameter | 2 | | 3 | |
| Empirical formula | C₈₁H₉₈Au₂F₁₂N₄OP₄ | | C₈₂H₁₂₅Au₂F₁₂N₅P₄ | |
| CCDC number | 2251701 | | 2251702 | |
| Formula weight | 1924.89 | | 1926.71 | |
| Wavelength/Å | 0.71073 | | 0.71073 | |
| Temperature/K | 304(2) | | 293(2) | |
| Crystal symmetry | Monoclinic | | Monoclinic | |
| Space group | C12/c1 | | P-1 | |
| Cell lengths (Å) | a = 44.5764(13), | | a = 15.3410(8), | |
| | b = 22.8282(7), | | b = 15.9107(8), | |
| | c = 18,8486(6) | | c = 20.3828(11) | |
| Cell Angles (°) | α = 90, | | α = 101.187(4), | |
| | β = 96.0350(10), | | β = 109.355(4), | |
| | γ = 90 | | γ = 94.215(4) | |
| Dₓ | 1.341 | g cm⁻³ | 1.405 | Mg m⁻³ |
| μ | 3.231 | mm⁻¹ | 3.355 | mm⁻¹ |

| | | |
|---|---|---|
| Z | 8 | 2 |
| Cell volume (Å³) | 19074.0(10) | 4553.5(4) |
| Theta range for data | 1.84 to 28.37° | |
| collection | | |
| Reflections collected | 91707 | |
| Independent reflections | 23226[R(int) = 0.0519] | |
| Goodness-of-fit on F² | 1.394 | 0.39, 1.000 |
| Final R indices | 15567 data, I > 2σ(I), | |
| | R1 = 0.0693, wR2 = 0.2081 | |
| | All data, R1 = 0.1049, | |
| | wR2 = 0.2214 | |
| R[F² > 2σ(F²)], wR(F²) S, | 0.844, 1.000 | 0.39, 1.000 |
| Tₘᵢₙ, Tₘₐₓ | | |
| H-atom treatment | H atoms treated by a mixture | |
| | of independent and | |
| | constrained refinement. | |
| Radiation source | | fine-focus sealed tube |
| Radiation monochromator | | graphite |
| (sin θ/λ)ₘₐₓ (Å⁻¹) | | 26.246 |

Suitable crystals of complex 3 were obtained as colorless rods from dichloromethane/ethanol solution. The X-ray data were collected at 173 K on a STOE IPSD 2 Image Plate Diffraction System (See: Stoe & Cie. (2009). X-Area & X-RED32. Stoe & Cie GmbH, Darmstadt, Germany, which is incorporated herein by reference in its entirety) connected with a two-circle goniometer and using MoK_{α} graphite monochromator (λ=0.71073 Å). The structure was solved by the SHELXS-2014 program (See: Sheldrick, G. M. A Short History of SHELX Acta Cryst A 2008, 64, 112-122, which is incorporated herein by reference in its entirety). The refinement and further calculations were carried out with SHELXL-2014 (See: Stoe & Cie. (2009). X-Area & X-RED32. Stoe & Cie GmbH, Darmstadt, Germany, which is incorporated herein by reference in its entirety). The non-hydrogen atoms were refined using a least-squares matrix on F2. A semi-empirical absorption correction was applied using the MUL scan ABS routine in PLATON (See: Spek, A. L. Structure Validation in Chemical Crystallography. Acta Cryst D 2009, 65, 148-155, which is incorporated herein by reference in its entirety). The crystal structure and crystal packing were drawn using Mercury software (See: Macrae, C. F.; Bruno, I. J.; Chisholm, J. A.; Edgington, P. R.; McCabe, P.; Pidcock, E.; Rodriguez-Monge, L.; Taylor, R.; Streek, J. van de; Wood, P. A. Mercury CSD 2.0 New Features for the Visualization and Investigation of Crystal Structures. J Appl Cryst 2008, 41, 466-470, which is incorporated herein by reference in its entirety). The structure refinement and the crystal data for complex 3 are also shown in Table 1.

### Example 6: Gold Complex Composition

Gold(I) complex was dissolved in dimethyl sulfoxide (DMSO) to a concentration of 10 mM. The exact amount of DMSO necessary to dissolve the compound was used as a negative control in all experiments. Cisplatin (Teva) was a surplus drug from the clinical pharmacy of CRO Aviano.

### Example 7: Cell Lines and Culture Conditions

The human epithelial ovarian cancer A2780 cell line and its cisplatin-resistant clone A2780cis were obtained from Sigma-Aldrich (Milano, Italy). A2780cis cells were continuously exposed to 1 μM cisplatin to maintain cisplatin resistance. A549 cells (Human non-small cell lung cancer), MCF7 (metastatic breast cancer), PC3 (highly aggressive metastatic prostate cancer), and MG63 (osteosarcoma) were obtained from the American Type Culture Collection (ATCC). Cells were authenticated in laboratory using the PowerPlex 16 HS System. A2780, A2780cis, and PC3 cells were cultured in RPMI-1640 medium; MG63 and MCF7 cells were cultured in DMEM, and A549 cells were cultured in F12 NUT-MIX. The culture medium contained about 10% fetal bovine serum (FBS) (Gibco, Thermo Fisher Scientific, Monza, Italy), about 1% (v/v) of penicillin (10,000 units/mL)-streptomycin (10 mg/mL), and about 1% (v/v) L-glutamine (200 mM) (all from Sigma-Aldrich). Cells were cultured at 37° C. in a 5% CO₂, fully humidified atmosphere.

### Example 8: Cytotoxicity Assay

2.5×10³ (PC3, MG63, MCF7) or 4.0×10³ (A2780, A2780cis, A549) cells were seeded in 96-well microplates in 100 μL culture medium. Cells, allowed to adhere for 24 h, were then treated in triplicate with increasing concentrations of gold compounds. Cisplatin was included as a reference drug. Cell viability was evaluated using 3-(4,5-dimethylthiazol-2-yl)-2,5-diphenyltetrazolium bromide (MTT) assay (Sigma-Aldrich) after 72 h of treatment. Half maximal inhibitory concentrations (IC₅₀), IC₂₅, and IC₇₅ were calculated using the CalcuSyn software version 2 (Biosoft, Ferguson, MO, USA) (See: Chou, T. C.; Talalay, P. Quantitative Analysis of Dose-Effect Relationships: The Combined Effects of Multiple Drugs or Enzyme Inhibitors. Adv. Enzyme Regul. 1984, 22, 27-55, which is incorporated herein by reference in its entirety). Fold resistance was calculated as the ratio of the IC₅₀ of the A2780cis to that of the parental cell line A2780. Alternatively, cell viability was assayed by crystal violet staining (See: Feoktistova, M.; Geserick, P.; Leverkus, M. Crystal Violet Assay for Determining Viability of Cultured Cells. Cold Spring Harb Protoc 2016, 2016, pdb.prot087379, which is incorporated herein by reference in its entirety). Alternatively, after drug treatment, the culture Medium was removed, cells washed, fixed, and stained, and the Crystal Violet solution lasted 20 minutes (See: Sulaiman, A. A. A.; Casagrande, N.; Borghese, C.; Corona, G.; Isab, A. A.; Ahmad, S.; Aldinucci, D.; Altaf M. Design, Synthesis, and Preclinical Activity in Ovarian Cancer Models of New Phosphanegold(I)—N-Heterocyclic Carbene Complexes. J Med Chem 2022, 65, 14424-14440, which is incorporated herein by reference in its entirety).

### Example 9: 3D-A549-MCTS Formation and Viability

To obtain multiple spheroids, 10.0×10⁴ A549 cells were seeded in non-adherent conditions on poly-HEMA coated 96 wells (final volume, 200 μL) for four days. After drug treatment (72 h), the cell viability was determined using PrestoBlue Cell Viability Reagent (Thermo Fisher Scientific, Frederick, MD, USA) (See: Casagrande, N.; Celegato, M.; Borghese, C.; Mongiat, M.; Colombatti, A.; Aldinucci, D. Preclinical Activity of the Liposomal Cisplatin Lipoplatin in Ovarian Cancer. Clin Cancer Res 2014, 20, 5496-5506, which is incorporated herein by reference in its entirety).

### Example 10: Flow Cytometry

2.0×10⁵ A549 cells were seeded in 6-well plates in a complete medium and treated with the complex 3. Assay results were detected by flow cytometry on a BD FACSCanto II flow cytometer. Data were analyzed using BD FACSDiva v.8.0.1 software (BD Biosciences) unless otherwise indicated. Tumor cells were treated for 72 h with complex 3, then stained for 15 minutes with FITC Annexin-V (Thermo Fisher Scientific) and 7AAD (BD Pharmingen, Milan, Italy). Activated caspase-3,7 was detected using the CaspaTag Caspase-3,7 assay (Merck Millipore) after an incubation of 24 h with complex 3. For B-cell lymphoma/leukemia-2 protein (Bcl-2) and Bcl-2 associated x protein (Bax) analysis, cells were treated for 72 h with C3, then fixed and permeabilized (FIX & PERM Cell Fixation & Cell Permeabilization Kit, Life Technologies). The cells were incubated with fluorescein isothiocyanate (FITC)-conjugated mouse anti-human Bcl-2 (clone 124) (DAKO Cytomation, Milan, Italy) or Mouse anti-human BAX (Clone 3/Bax) (RUO) (BD pharmingen). Bax was revealed with Alexa 488 Fluor-conjugated anti-mouse secondary antibody (Molecular probes). To evaluate mit-ROS generation, cells were cultured for 48 h with complex 3. Then, they were stained with 5 μM MitoSOX Red Mitochondrial Superoxide Indicator (Thermo Fisher Scientific) in a working solution for 30 min at 37° C. The distribution of cells in different cell cycle phases was quantified using ModFit LT 4.0 software (Verity Software House, Topsham, ME, USA) (See: Sulaiman, A. A. A.; Casagrande, N.; Borghese, C.; Corona, G.; Isab, A. A.; Ahmad, S.; Aldinucci, D.; Altaf M. Design, Synthesis, and Preclinical Activity in Ovarian Cancer Models of New Phosphanegold(I)—N-Heterocyclic Carbene Complexes. J Med Chem 2022, 65, 14424-14440, which is incorporated herein by reference in its entirety). A549 cells were treated for 24 h with complex 3. Then, the surface expression of CD44 was analyzed with FITC-conjugated anti-CD44 (Becton Dickinson, Italia), CD133 with AC133-PE, (130-080-801, Miltenyi Biotec S.r.l. Bologna, Italy), NOTCH1 with anti-Notch1-PE (R&D System), and the enzymatic activity of ALDH1 with Aldeflour (Stem Cell Technologies, Inc., Cambridge, MA, USA) (See: Casagrande, N.; Borghese, C.; Agostini, F.; Durante, C.; Mazzucato, M.; Colombatti, A.; Aldinucci, D. In Ovarian Cancer Multicellular Spheroids, Platelet Releasate Promotes Growth, Expansion of ALDH+ and CD133+ Cancer Stem Cells, and Protection against the Cytotoxic Effects of Cisplatin, Carboplatin and Paclitaxel. Int J Mol Sci 2021, 22, 3019, which is incorporated herein by reference in its entirety).

### Example 11: TrxR, 20S Proteasome, and NF-kB Activity

Cells were treated with complex 3 (24 h) and then lysed in 50 mM Tris-HCl pH 7.6, 0.1% Triton X-100, 0.9% NaCl to obtain the TrxR activity. TrxR (EC 1.8.1.9) was assayed using the Thioredoxin Reductase Assay Kit (Sigma-Aldrich). Enzyme activity was determined by reading the absorbance at 412 nm using a spectrophotometer (Biomate 3 Thermo Spectronic, Thermo Electronic Corporation, Monza, Italy) and normalized to the protein concentration, determined with the Bio-Rad protein assay (Protein Assay Dye Reagent Concentrate, Bio-Rad Laboratories, Segrate, Italy).

Cells were treated with complex 3 (4 h), and then cells were lysed in 50 mM Tris-HCl pH 7.6, 0.1% Triton X-100, 0.9% NaCl to obtain the Proteasome activity (EC 3.4.25.1). The proteasome activity was tested using the 20S Proteasome Activity Assay kit APT280 (Merck Millipore) and a computer-interfaced GeniusPlus microplate reader (Tecan Trading AG, Switzerland). The activity was normalized to the protein concentration and expressed as a percentage of control (untreated cells).

Cells were treated with complex 3 (12 h), collected, and nuclear protein was extracted. Briefly, cells were lysed with buffer A (10 mM HEPES (pH 7.9), 10 mM KCl, 1.5 mM MgCl₂, 0.5 mM dithiothreitol, 0.05% NP-40, 0.5 mM PMSF, 1 mM Na₃VO₄, 1 mM NaF) on ice for 30 min, to obtain the NF-kB p65 active form. Samples were centrifuged at 3,000 rpm for 10 min at 4° C. Pellets (nuclear fraction) were re-suspended in ice-cold extraction buffer B (5 mM HEPES (pH 7.9), 300 mM NaCl, 0.2 M EDTA, 1.5 mM MgCl₂, 25% (vol/vol) glycerol, 0.5 mM dithiothreitol, 0.5 mM PMSF, 1 mM Na₃VO₄, 1 mM NaF) and incubated on ice for 30 min. Samples were centrifuged at 13,000 rpm for 20 min at 4° C., and the supernatant was taken as the nuclear extract. Protein concentration was determined using the Bio-Rad Bradford protein assay. NF-kB DNA binding activity was analyzed using the Transcription Factor Kit for NF-kB p65 (Thermo Fisher Scientific). Data were normalized to that in A549 untreated cells.

### Example 12: Statistical Analysis

Statistical analysis was done using GraphPad Prism version 6.0 software (GraphPad, La Jolla, USA). Students' t-tests were used to compare the two groups. One-way ANOVA followed by Dunnett's test was used to compare each of a number of treatments with a single control. A P-value <0.05 was considered significant.

### Example 13: Results

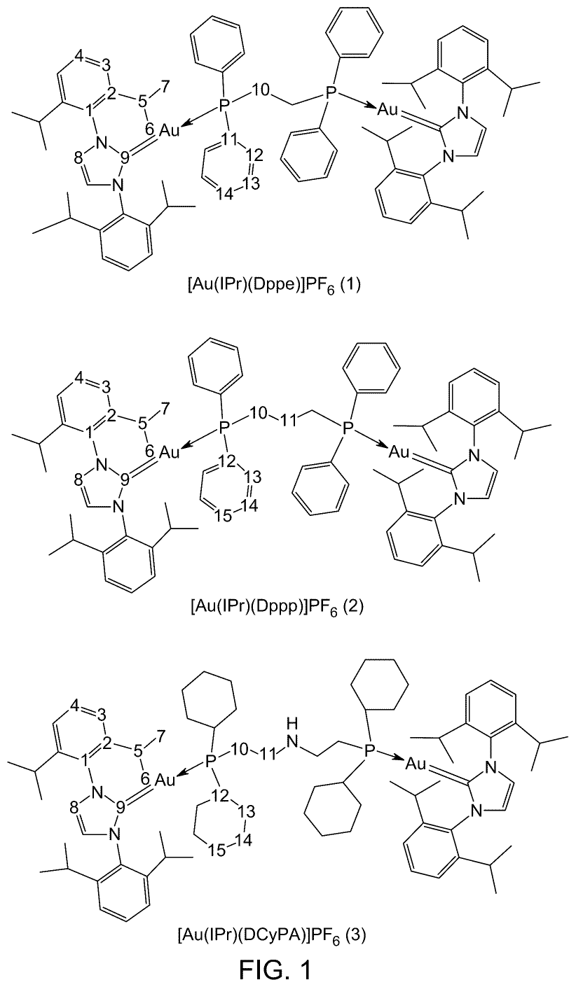

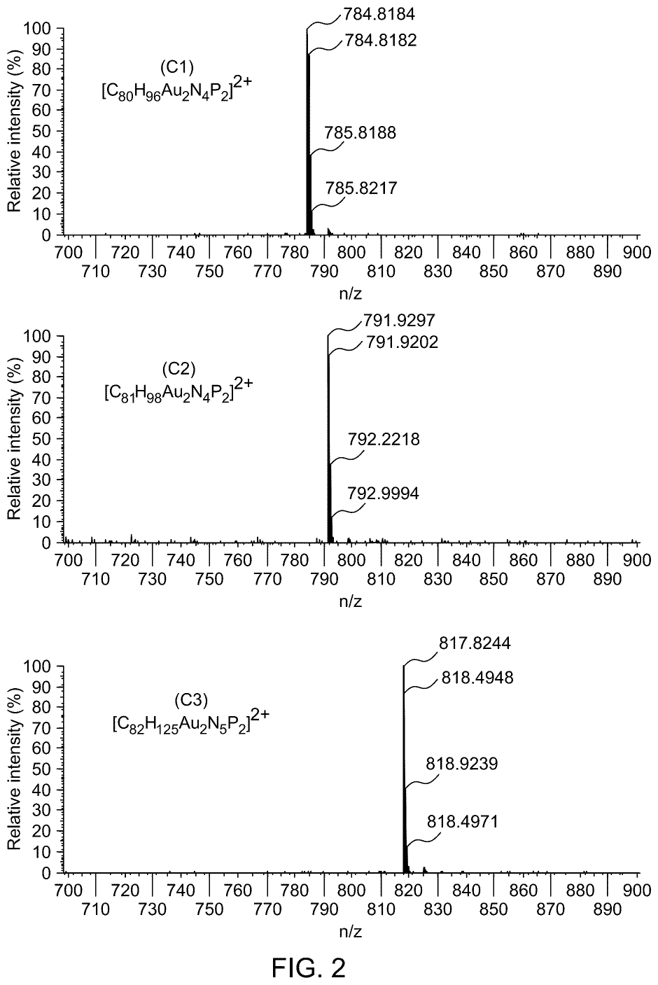

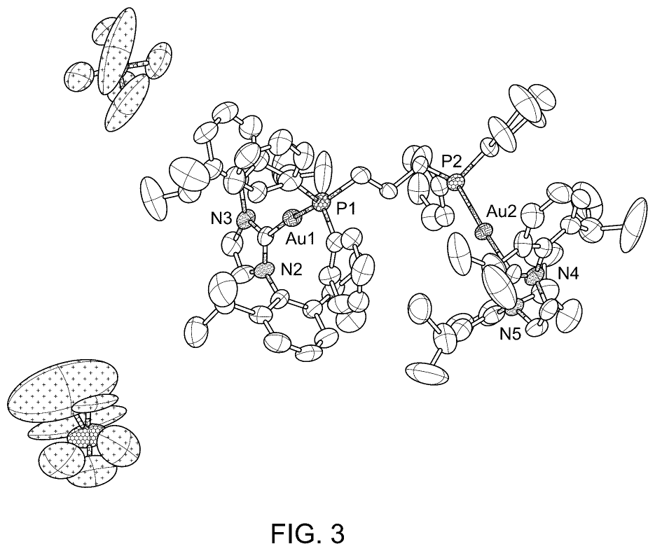

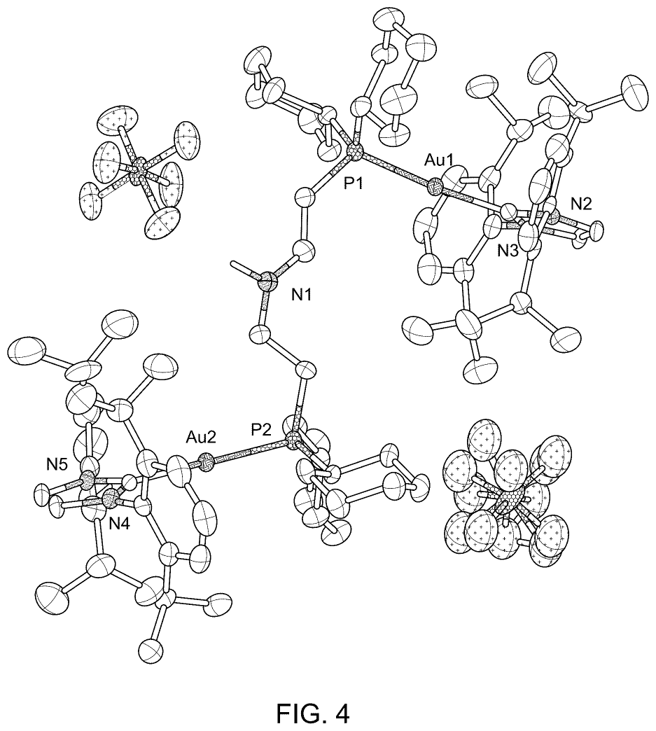

Gold(I) complexes (1-3) containing a carbene (1,3-bis(2,6-di-isopropylphenyl)imidazol-2-ylidene (IPr)) and diphosphane ligands [bis(1,2-diphenylphosphano)ethane (Dppe) (complex 1), bis(1,3-diphenylphosphano)propane (Dppp) (complex 2) and bis[2-(dicyclohexylphosphano)ethyl]amine (DCyPA)](complex 3) (FIG. 1), were prepared and characterized by elemental analysis, ESI-MS, IR and NMR spectroscopic methods. The preparation of complexes (1-3) was carried out in a two-step process. In the first step, the precursor complex chlorido(1,3-Bis(2,6-di-isopropylphenyl)imidazol-2-ylidene) gold(I), [Au(IPr)Cl] was reacted with AgPF₆ to replace Cl⁻ by a weakly coordinating ion, PF₆⁻. Adding a half equivalent of diphosphane to this mixture yielded the desired complexes. The synthesized complexes include: [Au(IPr)(Dppe)](PF₆)₂(1), [Au(IPr)(Dppp)](PF₆)₂(2), [Au(IPr)(DCyPA)](PF₆)₂(3); where Dppe=bis(1,2-diphenylphosphano)ethane, Dppp=bis(1,3-diphenylphosphano)propane and DCyPA=bis[2-(dicyclohexylphosphano) ethyl]amine. The composition of the complexes (1-3) was confirmed by elemental analysis. The compounds were further characterized by mass spectrometry (FIG. 2), mid-FT-IR, and NMR (¹H, ¹³C, and ³¹P) spectroscopy. The structure of complexes 2 (FIG. 3) and 3 (FIG. 4) was determined by single crystal X-ray diffraction analysis (Tables 1).

The high-resolution ESI positive mass spectral analysis of all three purified gold(I) complexes indicated that the complexes are preferentially present in solution as double positive charge species (FIG. 2). The m/z signals and the isotope pattern for the (1-3) gold cation complexes differ less than 2.6 milli atomic mass unit (a.m.u.) from the expected value confirming their elementary formula composition.

In the IR spectra of free ligands and [Au(IPr)Cl], weak absorbance, about 3037 cm⁻¹, is assigned to the ═C—H stretching absorptions of the phenyl ring. This absorbance was shifted towards a higher frequency (3172-3175 cm⁻¹) in the complexes. The aliphatic C—H was observed in the regions of 2960 and 2850 cm⁻¹. The ν(C═C) bands appeared around 1450 cm⁻¹. The C—P stretching vibrations of phosphanes were observed near 1100 cm 1. In complex 3, the ν(N—H) mode was detected at 3333 cm⁻¹, which for the free ligand appeared at 3258 cm⁻¹. A sharp band at 839 cm⁻¹ shows the presence of the P—F bond of PF₆ ion (See: Sulaiman, A. A. A.; Casagrande, N.; Borghese, C.; Corona, G.; Isab, A. A.; Ahmad, S.; Aldinucci, D.; Altaf M Design, Synthesis, and Preclinical Activity in Ovarian Cancer Models of New Phosphanegold(I)—N-Heterocyclic Carbene Complexes. J Med Chem 2022, 65, 14424-14440; Sulaiman, A. A. A.; Ahmad, S.; Hashimi, S. M.; Alqosaibi, A. I.; Peedikakkal, A. M. P.; Alhoshani, A.; Alsaleh, N. B.; Isab, A. A. Novel Dinuclear Gold(I) Complexes Containing Bis(Diphenylphosphano)Alkanes and (Biphenyl-2-Yl)(Di-Tert-Butyl)Phosphane: Synthesis, Structural Characterization and Anticancer Activity. New J. Chem. 2022, 46, 16821-16831; and Abogosh, A. K.; Alghanem, M. K.; Ahmad, S.; Al-Asmari, A.; As Sobeai, H. M.; Sulaiman, A. A. A.; Fettouhi, M.; Popoola, S. A.; Alhoshani, A.; Isab, A. A. A Novel Cyclic Dinuclear Gold(I) Complex Induces Anticancer Activity via an Oxidative Stress-Mediated Intrinsic Apoptotic Pathway in MDA-MB-231 Cancer Cells. Dalton Trans 2022, 51, 2760-2769).

The ¹H NMR chemical shifts of free ligands and their complexes (1-3) are given in Table 2. The ¹H NMR spectra of all three complexes showed the expected six characteristic signals of the carbene (IPr) moiety of the complexes (See: Sulaiman, A. A. A.; Kalia, N.; Bhatia, G.; Kaur, M.; Fettouhi, M.; Altaf M.; Baig, N.; Kawde, A.-N.; Isab, A. A. Cytotoxic Effects of Gold(I) Complexes against Colon, Cervical and Osteo Carcinoma Cell Lines: A Mechanistic Approach. New J. Chem. 2019, 43, 14565-14574; Altaf M.; Monim-ul-Mehboob, M.; Seliman, A. A. A.; Isab, A. A.; Dhuna, V.; Bhatia, G.; Dhuna, K. Synthesis, X-Ray Structures, Spectroscopic Analysis and Anticancer Activity of Novel Gold(I) Carbene Complexes. Journal of Organometallic Chemistry 2014, 765, 68-79; Seliman, A. A. A.; Altaf M.; Odewunmi, N. A.; Kawde, A.-N.; Zierkiewicz, W.; Ahmad, S.; Altuwaijri, S.; Isab, A. A. Synthesis, X-Ray Structure, DFT Calculations and Anticancer Activity of a Selenourea Coordinated Gold(I)-Carbene Complex. Polyhedron 2017, 137, 197-206; Seliman, A. A. A.; Altaf M.; Onawole, A. T.; Ahmad, S.; Ahmed, M. Y.; Al-Saadi, A. A.; Altuwaijri, S.; Bhatia, G.; Singh, J.; Isab, A. A. Synthesis, X-Ray Structures and Anticancer Activity of Gold(I)-Carbene Complexes with Selenones as Co-Ligands and Their Molecular Docking Studies with Thioredoxin Reductase. Journal of Organometallic Chemistry 2017, 848, 175-183; Seliman, A. A. A.; Altaf M; Onawole, A. T.; Al-Saadi, A.; Ahmad, S.; Alhoshani, A.; Bhatia, G.; Isab, A. A. Synthesis, X-Ray Structure and Cytotoxicity Evaluation of Carbene-Based Gold(I) Complexes of Selenones. Inorganica Chimica Acta 2018, 476, 46-53, each of which is incorporated herein by reference in their entireties).

TABLE 2
¹H NMR chemical shifts (ppm) for free ligands
and gold(I) complexes 1-3 in CDCl₃/DMSO-d₆.

| Ligand/ | | | | | | | | | | | | |
|---|---|---|---|---|---|---|---|---|---|---|---|---|
| complex | H3 | H4 | H5 | H6 | H7 | H8 | H10 | H11 | H12 | H13 | H14 | H15 |
| [Au(IPr)Cl | 7.55 | 7.39 | 2.46 | 1.33 | 1.21 | 7.98 | — | — | — | — | — | — |
| Dppe | — | — | — | — | — | — | 2.10 | — | 7.30 | 7.32 | — | — |
| Complex 1 | 7.53 | 7.31 | 2.40 | 1.14 | 0.99 | 7.75 | 1.78 | — | 6.74 | 7.47 | 7.22 | — |
| Dppp | — | — | — | — | — | — | 1.62 | 2.36 | 7.30 | 7.36 | — | — |
| Complex 2 | 7.61 | 7.40 | 2.40 | 1.22 | 0.97 | 8.02 | 1.34 | 1.14 | 6.93 | 7.69 | 7.50 | 7.18 |
| DCyPA | — | — | — | — | — | — | 1.80 | 2.97 | 2.14 | 1.76 | 1.56 | — |
| Complex 3 | 7.42 | 7.24 | 2.47 | 1.23 | 1.21 | 7.49 | 1.70 | 2.23 | 1.62 | 1.49 | 1.43 | 1.10 |

In the spectra of complexes 1 and 2, the aromatic C—H chemical shifts of Dppe and Dppp ligands were observed in the range of 6.7-7.7 ppm. In comparison, the methylene protons resonated around 1 ppm. In complex 3, the P(CH) protons of the cyclohexyl groups were detected near 1.7 ppm. The signal for the N—H proton was found at the most downfield position (3.68 ppm) of the aliphatic region. The remaining protons of the cyclohexyl part resonated between 1-2 ppm. The ¹³C NMR chemical shifts for free ligands and their complexes are summarized in supplemental Table 3. In the ¹³C NMR spectra of the prepared gold(I) complexes (1-3), downfield shifts were observed in the C═Au resonances with respect to the starting complex, [Au(IPr)Cl](188.9, 189.0, 192.2 ppm respectively vs 175.3 ppm). The C—N aromatic carbon appeared at the second-most downfield position (near 145 ppm). The C—P resonances shifted upfield, indicating the coordination of diphosphanes. The rest of the peaks were in accordance with the structures of the ligands (See: Sulaiman, A. A. A.; Casagrande, N.; Borghese, C.; Corona, G.; Isab, A. A.; Ahmad, S.; Aldinucci, D.; Altaf M. Design, Synthesis, and Preclinical Activity in Ovarian Cancer Models of New Phosphanegold(I)—N-Heterocyclic Carbene Complexes. J Med Chem 2022, 65, 14424-14440; Sulaiman, A. A. A.; Ahmad, S.; Hashimi, S. M.; Alqosaibi, A. I.; Peedikakkal, A. M. P.; Alhoshani, A.; Alsaleh, N. B.; Isab, A. A. Novel Dinuclear Gold(I) Complexes Containing Bis(Diphenylphosphano)Alkanes and (Biphenyl-2-Yl)(Di-Tert-Butyl)Phosphane: Synthesis, Structural Characterization and Anticancer Activity. New J. Chem. 2022, 46, 1682116831; and Abogosh, A. K.; Alghanem, M. K.; Ahmad, S.; Al-Asmari, A.; As Sobeai, H. M.; Sulaiman, A. A. A.; Fettouhi, M; Popoola, S. A.; Alhoshani, A.; Isab, A. A. A Novel Cyclic Dinuclear Gold(I) Complex Induces Anticancer Activity via an Oxidative Stress-Mediated Intrinsic Apoptotic Pathway in MDA-MB-231 Cancer Cells. Dalton Trans 2022, 51, 2760-2769; Sulaiman, A. A. A.; Kalia, N.; Bhatia, G.; Kaur, M.; Fettouhi, M.; Altaf M.; Baig, N.; Kawde, A.-N.; Isab, A. A. Cytotoxic Effects of Gold(I) Complexes against Colon, Cervical and Osteo Carcinoma Cell Lines: A Mechanistic Approach. New J Chem. 2019, 43, 14565-14574; Altaf M.; Monim-ul-Mehboob, M.; Seliman, A. A. A.; Isab, A. A.; Dhuna, V.; Bhatia, G.; Dhuna, K. Synthesis, X-Ray Structures, Spectroscopic Analysis and Anticancer Activity of Novel Gold(I) Carbene Complexes. Journal of Organometallic Chemistry 2014, 765, 68-79; Seliman, A. A. A.; Altaf M; Odewunmi, N. A.; Kawde, A.-N.; Zierkiewicz, W.; Ahmad, S.; Altuwaijri, S.; Isab, A. A. Synthesis, X-Ray Structure, DFT Calculations and Anticancer Activity of a Selenourea Coordinated Gold(I)-Carbene Complex. Polyhedron 2017, 137, 197-206; Seliman, A. A. A.; Altaf M; Onawole, A. T.; Ahmad, S.; Ahmed, M. Y.; Al-Saadi, A. A.; Altuwaijri, S.; Bhatia, G.; Singh, J.; Isab, A. A. Synthesis, X-Ray Structures and Anticancer Activity of Gold(I)-Carbene Complexes with Selenones as Co-Ligands and Their Molecular Docking Studies with Thioredoxin Reductase. Journal of Organometallic Chemistry 2017, 848, 175-183; Seliman, A. A. A.; Altaf M; Onawole, A. T.; Al-Saadi, A.; Ahmad, S.; Alhoshani, A.; Bhatia, G.; Isab, A. A. Synthesis, X-Ray Structure and Cytotoxicity Evaluation of Carbene-Based Gold(I) Complexes of Selenones. Inorganica Chimica Acta 2018, 476, 46-53, each of which is incorporated herein by reference in their entireties).

TABLE 3
¹³C NMR chemical shifts (ppm) of free ligands and gold(I) complexes 1-3 in CDCl₃/DMSO-d₆.

| Ligand/ | | | | | | | | | | | | | | |
|---|---|---|---|---|---|---|---|---|---|---|---|---|---|---|
| complex | C1 | C2 | C3 | C4 | C5 | C6/7 | C8 | Au═C | C10 | C11 | C12 | C13 | C14 | C15 |
| [Au(IPr)Cl | 145.5 | 133.9 | 130.7 | 123.0 | 28.8 | 24.0, | 124.2 | 175.3 | — | — | — | — | — | — |
| | | | | | | 24.5 | | | | | | | | |
| Dppe | — | — | — | — | — | — | — | — | 23.7 | 137.9 | 132.6 | 128.3 | 125.1 | — |
| Complex 1 | 145.4 | 131.0 | 133.0 | 124.1 | 28.5 | 23.7, | 124.1 | 188.9 | 23.0 | 126.3 | 132.4 | 129.7 | 125.4 | — |
| | | | | | | 24.5 | | | | | | | | |
| Dppp | — | — | — | — | — | — | — | — | 29.6 | 22.4 | 138.6 | 132.7 | 128.4 | 129.1 |
| Complex 2 | 145.2 | 130.9 | 133.0 | 124.2 | 28.3 | 24.1, | 124.2 | 189.0 | 26.4 | 19.1 | 128.0 | 132.5 | 129.5 | 125.6 |
| | | | | | | 23.4 | | | | | | | | |
| DCyPA | — | — | — | — | — | — | — | — | 33.0 | 46.9 | 32.9 | 30.0 | 28.8 | 26.3 |
| Complex 3 | 145.7 | 131.0 | 133.2 | 124.5 | 28.8 | 24.0, | 124.2 | 192.2 | 33.2 | 47.0 | 26.4 | 29.5 | 28.6 | 25.4 |
| | | | | | | 24.8 | | | | | | | | |

The ³¹P NMR chemical shifts (ppm) for free ligands and gold(I) complexes (1-3) are given in Table 4. In ³¹P NMR spectra of complexes, the resonances for the prepared complexes were observed downfield (31.88-41.79 ppm) with respect to the free phosphane ligands (−14.26, −19.08, −10.40 ppm) respectively. The downfield shift was related to the transfer of electron density from phosphorus to gold(I) ions.

TABLE 4
³¹P NMR chemical shifts of free ligands
and gold(I) complexes 1-3 in DMSO-d₆.

| Compound | δ ³¹P (ppm) |
|---|---|
| Dppe | −14.26 |
| C1 | 35.56 |
| Dppp | −19.08 |
| C2 | 31.88 |
| DCyPA | −10.40 |
| C3 | 41.79 |

The selected bond lengths and bond angles of complex 2 are in Table 5. The structure refinement and the crystal data for complex 2 are shown in Table 1. The complex consists of dinuclear [{Au(IPr)}₂(Dppp)]²⁺ cations and PF₆⁻ counter ions. Each gold(I) ion is coordinated by a carbene (IPr) carbon atom and a phosphorous atom of bridging diphosphane (Dppp) ligand. The geometry around the gold(I) atom is described as distorted linear coordination with a C—Au—P angle of 174.2(2)°-175.1 (2°). The Au—C bond distance was in the range of 2.023 (7)-2.025 (7).

TABLE 5
Selected bond lengths and bond angles for complex 2.

| | |
|---|---|
| Bond Length (Å) | Bond Angles (°) |

| | | | |
|---|---|---|---|
| Au1—P1 | 2.289 (2) | C16—Au1—P1 | 174.2 (2) |
| Au1—P2 | 2.270 (2) | C67—Au2—P2 | 175.1 (2) |
| Au2—C16 | 2.025 (7) | C10—P1—Au1 | 110.7 (3) |
| Au2—C67 | 2.023 (7) | C2—P1—Au1 | 117.5 (3) |
| P1—C10 | 1.820 (8) | C4—P1—Au1 | 112.5 (3) |
| P1—C2 | 1.835 (8) | C49—P2—Au2 | 113.2 (3) |
| P2—C43 | 1.810 (8) | C3—P2—Au2 | 117.2 (3) |
| P2—C49 | 1.789 (8) | C43—P2—Au2 | 106.8 (3) |

The molecular structure of complex 3 is illustrated in FIG. 4. The selected bond lengths and bond angles of complex 3 are in Table 6. The structure refinement and the crystal data for complex 3 are shown in Table 1. The complex consists of dinuclear [{Au(IPr)}₂(Diphosphane)]²⁺ cations and PF₆⁻ counter ions. Each gold(I) ion is coordinated by a carbene (IPr) carbon atom and phosphorus atom of bridging diphosphane (DCyPA) ligand.

TABLE 6
Selected bond lengths and bond angles for complex 3

| | |
|---|---|
| Bond Length (Å) | Bond Angles (°) |

| | | | |
|---|---|---|---|
| Au1—P1 | 2.285 (1) | P1—Au1—C5 | 175.0 (1) |
| Au1—C5 | 2.024 (5) | P2—Au2—C32 | 175.0 (1) |
| Au2—P2 | 2.286 (1) | Au1—P1—C1 | 116.5 (2) |
| Au2—C32 | 2.023 (5) | Au1—P1—C59 | 110.6 (2) |
| P1—C1 | 1.823 (6) | Au1—P1—C65 | 110.0 (2) |
| P1—C59 | 1.836 (4) | Au2—P2—C4 | 112.8 (2) |
| P2—C4 | 1.828 (5) | Au2—P2—C71 | 110.4 (2) |
| P2—C71 | 1.831 (4) | Au2—P2—C77 | 112.7 (2) |

The geometry around the gold(I) atom is described as distorted linear coordination with a C—Au—P angle of 175.0(1°). The binding of Au(I) ions with the N—H moiety was not observed. The Au—C bond distance of 2.023(5) Å in 3 was in line with the value found for the related monophosphane complex (2.022(12) Å) containing a C—Au—P linkage (See: Sulaiman, A. A. A.; Casagrande, N.; Borghese, C.; Corona, G.; Isab, A. A.; Ahmad, S.; Aldinucci, D.; Altaf M Design, Synthesis, and Preclinical Activity in Ovarian Cancer Models of New Phosphanegold(I)—N-Heterocyclic Carbene Complexes. J Med Chem 2022, 65, 14424-14440; Rubbiani, R.; Can, S.; Kitanovic, L; Alborzinia, H.; Stefanopoulou, M.; Kokoschka, M.; Monchgesang, S.; Sheldrick, W. S.; Wolfi, S.; Ott, I. Comparative in Vitro Evaluation of N-Heterocyclic Carbene Gold(I) Complexes of the Benzimidazolylidene Type. J. Med. Chem. 2011, 54, 8646-8657; and Liu, W.; Bensdorf K.; Proetto, M; Abram, U.; Hagenbach, A.; Gust, R. NHC Gold Halide Complexes Derived from 4,5-Diarylimidazoles: Synthesis, Structural Analysis, and Pharmacological Investigations as Potential Antitumor Agents. J Med Chem 2011, 54, 8605-8615, each of which is incorporated herein by reference in their entireties). The Au—P bond distance, 2.285(1) Å was shorter than that in the corresponding complex of diphosphane without carbene, [Au((DCyPA)]₂(PF₆)₂ (2.313(1) Å)](See: Abogosh, A. K.; Alghanem, M. K.; Ahmad, S.; Al-Asmari, A.; As Sobeai, H. M.; Sulaiman, A. A. A.; Fettouhi, M.; Popoola, S. A.; Alhoshani, A.; Isab, A. A. A Novel Cyclic Dinuclear Gold(I) Complex Induces Anticancer Activity via an Oxidative Stress-Mediated Intrinsic Apoptotic Pathway in MDA-MB-231 Cancer Cells. Dalton Trans 2022, 51, 2760-2769, which is incorporated herein by reference in its entirety) as well as its close analogue, {Au[(bis-1,2-dicyclohexylphosphano)ethane]}₂(PF₆)₂(2.314(2) Å), resultinger distance indicated the better electron-donating capacity of carbene (IPr) that resulted in stronger π acceptance by the diphosphane (DCyPA). In the phosphane ligand, the P—C distances and the C—P—C or Au—P—C angles were in the range reported for other gold(I)-phosphane complexes (See: Sulaiman, A. A. A.; Casagrande, N.; Borghese, C.; Corona, G.; Isab, A. A.; Ahmad, S.; Aldinucci, D.; Altaf M Design, Synthesis, and Preclinical Activity in Ovarian Cancer Models of New Phosphanegold(I)—N-Heterocyclic Carbene Complexes. J Med Chem 2022, 65, 14424-14440; Sulaiman, A. A. A.; Ahmad, S.; Hashimi, S. M.; Alqosaibi, A. I.; Peedikakkal, A. M. P.; Alhoshani, A.; Alsaleh, N. B.; Isab, A. A. Novel Dinuclear Gold(I) Complexes Containing Bis(Diphenylphosphano)Alkanes and (Biphenyl-2-Yl)(Di-Tert-Butyl)Phosphane: Synthesis, Structural Characterization and Anticancer Activity. New J. Chem. 2022, 46, 16821-16831, Abogosh, A. K.; Alghanem, M. K.; Ahmad, S.; Al-Asmari, A.; As Sobeai, H. M.; Sulaiman, A. A. A.; Fettouhi, M.; Popoola, S. A.; Alhoshani, A.; Isab, A. A. A Novel Cyclic Dinuclear Gold(I) Complex Induces Anticancer Activity via an Oxidative Stress-Mediated Intrinsic Apoptotic Pathway in MDA-MB-231 Cancer Cells. Dalton Trans 2022, 51, 2760-2769, each of which is incorporated herein by reference in their entireties). The counter ion, PF₆⁻, was engaged in the C—H···F hydrogen bonding interactions with one of the methylene H atoms of the ligand. No Au—Au interactions were observed in either complex. The structure of complex 3 finds similarity in the gold(I) complexes of (biphenyl-2-yl)di-tert-butylphosphane (Bdbp) and diphosphane [Au₂(Bdbp)₂(Diphosphane)](PF₆)₂ (See: Sulaiman, A. A. A.; Ahmad, S.; Hashimi, S. M.; Alqosaibi, A. I.; Peedikakkal, A. M. P.; Alhoshani, A.; Alsaleh, N. B.; Isab, A. A. Novel Dinuclear Gold(I) Complexes Containing Bis(Diphenylphosphano)Alkanes and (Biphenyl-2-Yl)(Di-Tert-Butyl)Phosphane: Synthesis, Structural Characterization and Anticancer Activity. New J. Chem. 2022, 46, 16821-16831, each of which is incorporated herein by reference in their entireties). The same phosphane (DCyPA) in the absence of a carbene was reported to form a cyclic dinuclear complex, [Au((DCyPA)]₂(PF₆)₂ due to the bridging nature of diphosphane (See: Abogosh, A. K.; Alghanem, M. K.; Ahmad, S.; Al-Asmari, A.; As Sobeai, H. M.; Sulaiman, A. A. A.; Fettouhi, M.; Popoola, S. A.; Alhoshani, A.; Isab, A. A. A Novel Cyclic Dinuclear Gold(I) Complex Induces Anticancer Activity via an Oxidative Stress-Mediated Intrinsic Apoptotic Pathway in MDA-MB-231 Cancer Cells. Dalton Trans 2022, 51, 2760-2769, which is incorporated herein by reference in its entirety).

The growth inhibition potential of gold(I) complexes (1-3) was tested against a panel of different cancer cell lines, including A549, non-small cell lung cancer cells, PC3, androgen-resistant prostate cancer, MCF-7, breast cancer, and MG-63, osteosarcoma (Table 7) were used. For comparative purposes, cisplatin was included in this study. Cisplatin had relatively low potency on MCF-7 and MG-63 cells, with half maximal inhibitory concentrations (IC₅₀) >20 μM. In PC3 and A549 cells, cisplatin had an IC₅₀ of 3.3 μM and 6.2 μM, respectively. Gold(I) complexes (1-3) were more potent than cisplatin in all cell lines tested with IC₅₀, ranging from 0.13 μM to 0.43 μM (Table 7).

TABLE 7
Half-maximal inhibitory concentrations (IC₅₀) of cisplatin and gold(I)
complexes in A549 (lung cancer cells), PC3 (androgen-resistant prostate
cancer), MCF-7 (breast cancer), and MG-63 (osteosarcoma) cell lines.

| | IC₅₀ (μM) | | | | | |
|---|---|---|---|---|---|---|
| Compound | A549 | PC3 | MCF-7 | | MG-63 | |
| Cisplatin | 6.21 (0.72) | 3.32 (0.31) | 22.24 | (2.41) | 58.01 | (6.21) |
| Complex 1 | 0.22 (0.06) | 0.28 (0.02) | 0.17 | (0.01) | 0.31 | (0.05) |
| Complex 2 | 0.26 (0.04) | 0.25 (0.03) | 0.28 | (0.04) | 0.42 | (0.07) |
| Complex 3 | 0.13 (0.05) | 0.24 (0.02) | 0.43 | (0.06) | 0.16 | (0.03) |

¹ Mean (standard deviation, SD).

Complex 3 was the most potent compound in A549, PC3, and MG-63; complex 1 was in MCF-7 cells. The growth inhibition potential of the gold(I) complexes was also tested in the OvCa cell lines A2780 and its cisplatin-resistant clone A2780cis. In A2780 cells, gold(I) complexes (1-3) were more potent than cisplatin (IC₅₀ values ranging from 0.11 to 0.28 μM) (Table 8). The fold resistance (FR) (i.e., IC50 A2780cis/IC50 A2780) between the two cell lines was 6.93 for cisplatin and ranged from 1.18 to 2.19 for the gold complexes (1-3), thus excluding cross-resistance with cisplatin (Table 8).

TABLE 8
Half-maximal inhibitory concentrations (IC₅₀) of gold(I)
complexes and cisplatin in A2780 and A2780cis.

| | IC₅₀ (μM) | | | |
|---|---|---|---|---|
| | | | | Fold resistance |
| Compound | A2780 | A2780cis | | (A2780cis/A2780) |
| Cisplatin | 1.51 (0.12) | 10.41 | (0.97) | 6.93 |
| Complex 1 | 0.28 (0.02) | 0.33 | (0.06) | 1.18 |
| Complex 2 | 0.28 (0.03) | 0.45 | (0.05) | 1.61 |
| Complex 3 | 0.11 (0.01) | 0.24 | (0.03) | 2.19 |

¹ Mean (standard deviation, SD).

In vitro anti-proliferative activity of a series of seven [Au(IPr)(R₃P)]PF₆ complexes against A2780 and A2780cis OvCa cells was disclosed (See: Sulaiman, A. A. A.; Casagrande, N.; Borghese, C.; Corona, G.; Isab, A. A.; Ahmad, S.; Aldinucci, D.; Altaf M Design, Synthesis, and Preclinical Activity in Ovarian Cancer Models of New Phosphanegold(I)—N-Heterocyclic Carbene Complexes. J Med Chem 2022, 65, 14424-14440, which is incorporated herein by reference in its entirety). It was found that the tricyclohexylphosphane (Cy₃P) complex was an active compound. The [Au(IPr)(Cy₃P)]PF₆·CHCl₃, (complex 3) was more active in both A2780 and A2780cis cells, showing that the presence of cyclohexyl can confer high toxicity to gold(I) complexes.

### Example 14: Mechanism of Action of Complex 3 in A549 Cells

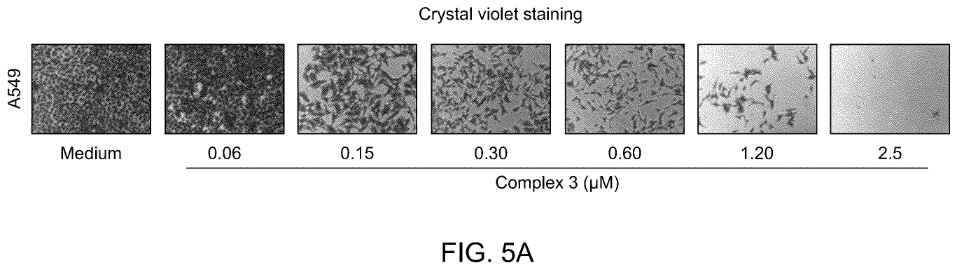

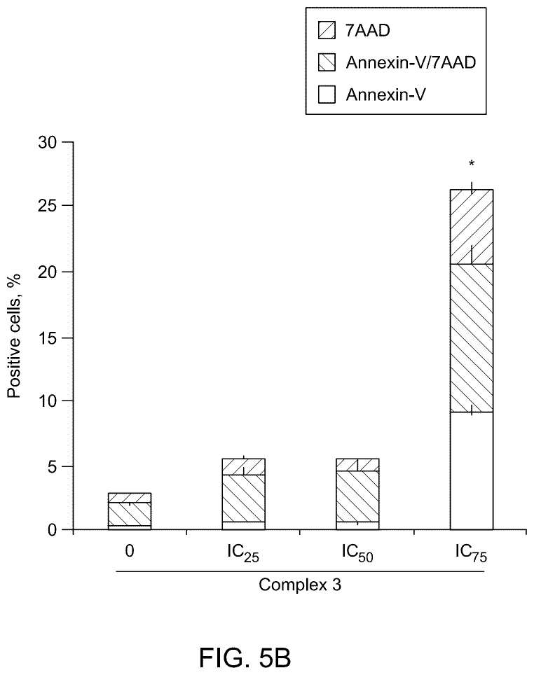

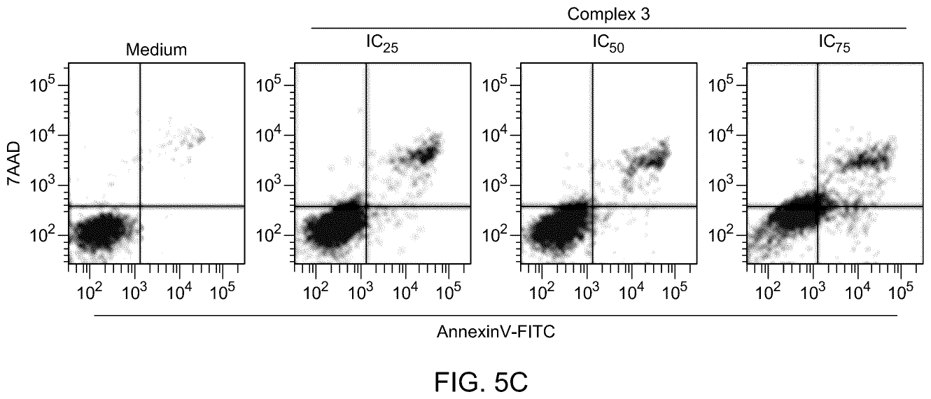

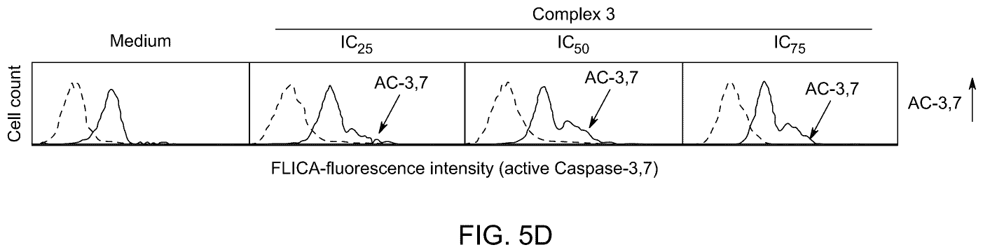

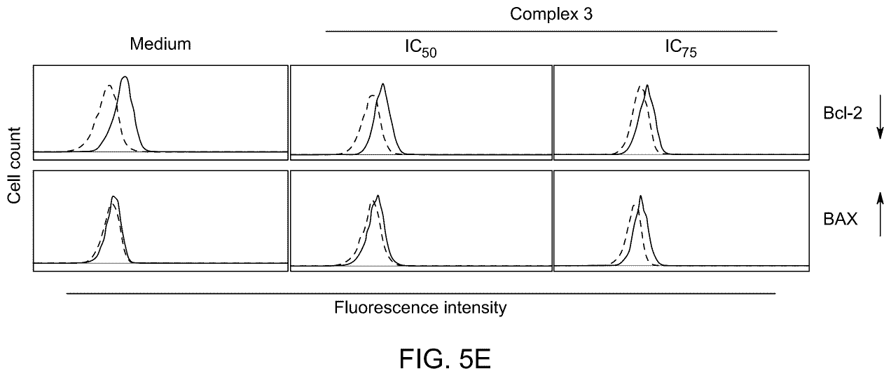

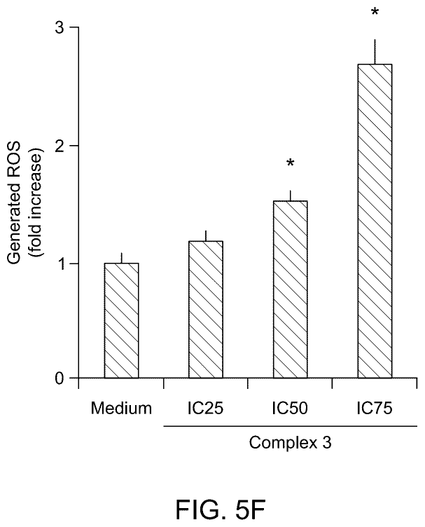

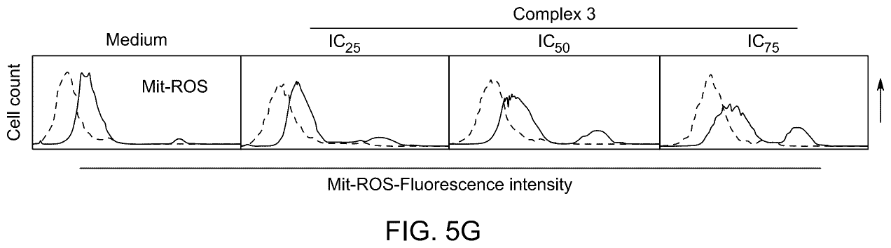

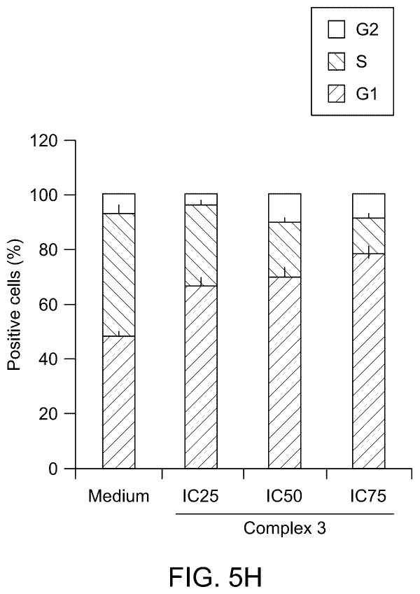

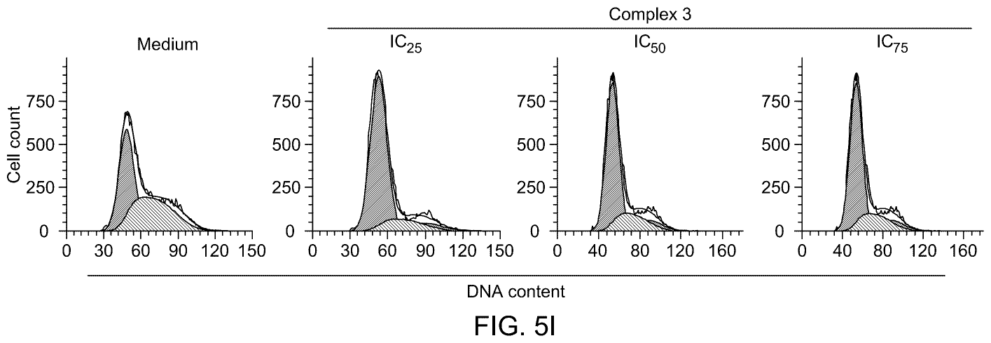

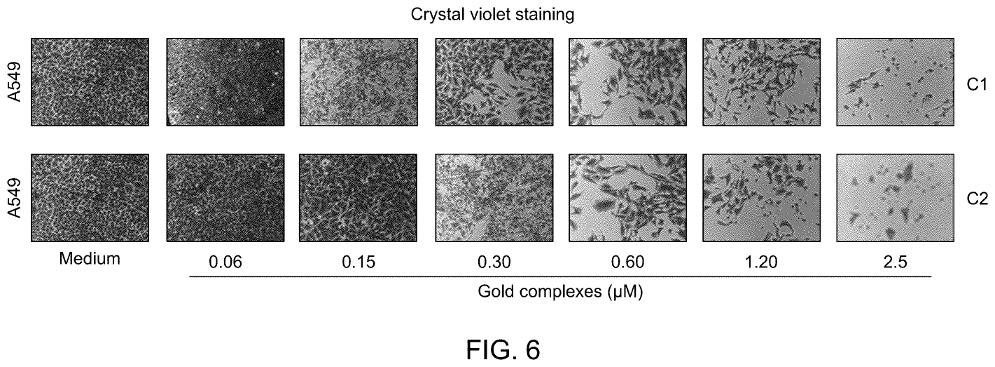

A549 lung cancer cells were used to determine the mechanism of action of complex 3, one of the potent among the new three gold complexes (Tables 7 and 8). The growth inhibition of A549 cells by complex 3 was further demonstrated using crystal violet staining (CV) (FIG. 5A). Representative images of A549 cells stained with CV after treatment with complex 3 (72 h) are shown in FIG. 5A and after treatment with complexes 1 and 2 in FIG. 6. Treatment of A549 cells with complex 3 (IC₂₅=0.06 μM, IC₅₀=0.13 μM, IC₇₅=0.60 μM) increased in a dose-dependent manner the percentage of Annexin-V-positive cells (early apoptosis) and of the double stained Annexin-V/7AAD-positive cells (late apoptosis) (FIG. 5B and FIG. 5C). Consistently, complex 3 activated caspase-3, 7 (FIG. 5D), down-regulated the anti-apoptotic BCL-2 and induced the pro-apoptotic BAX (FIG. 5E), showing the involvement of apoptosis in the complex 3 anticancer activity. Complex 3 increased mitochondrial reactive oxygen species (mit-ROS) (FIG. 5F and FIG. 5G). Bar charts of FIG. 5F reports the fold increase of mit-ROS positive cells. Representative flow cytometry histograms showing ROS levels after complex 3 treatment are shown in FIG. 5G.

Considering that cell cycle arrest can inhibit cell proliferation, the effect of complex 3 on cell cycle progression was tested (See: Matthews, H. K.; Bertoli, C.; de Bruin, R. A. M. Cell Cycle Control in Cancer. Nat Rev Mol Cell Biol 2022, 23, 74-88, which is incorporated herein by reference in its entirety). A549 cells were treated for 24 h with complex 3 (0.06-0.60 μM), and then cell cycle phases were analyzed by flow cytometry. It shows that complex 3 induced an accumulation in the G₀/G₁ phase of the cell cycle and a decrease in the S phase (FIG. 5H). Representative flow cytometry histograms of cell cycle phase modifications after treatment with complex 3 are shown in FIG. 5I. Apoptosis and cell cycle arrest are involved in the anti-cancer activity of complex 3.

### Example 15: Effects of Complex 3 on the Activity of the 20S-Proteasome, NF-kB, and TrxR

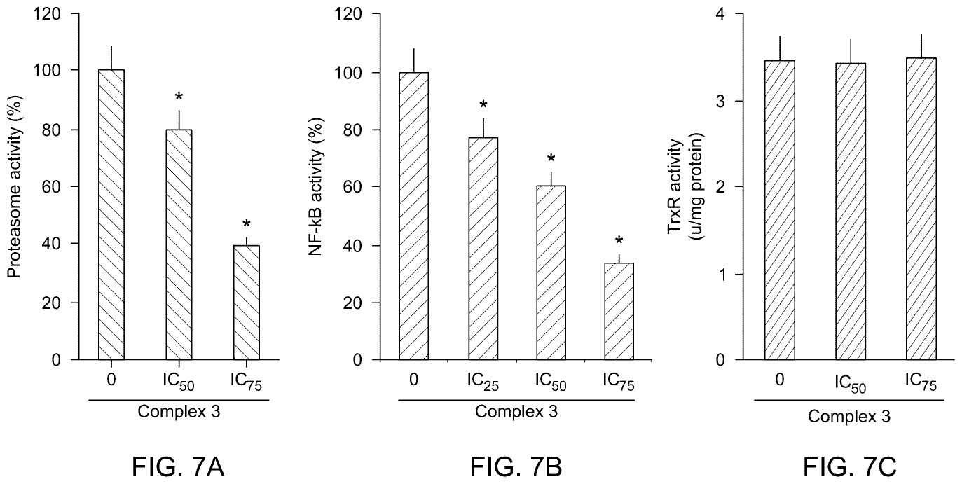

The proteasome, a target of several gold complexes, is fundamental for protein degradation. Its inhibitors can decrease cell survival pathways, inhibit NF-kB activity, increase ROS generation, and induce apoptosis. It was found that complex 3 inhibited in a dose-dependent manner both the 20S proteasome (FIG. 7A) and the activity of NF-kB (FIG. 7B), which are both upregulated in non-small cell lung cancer (NSCLC) tissues and associated with lung cancer progression and metastasis formation (See: NEMO- and Re/A-Dependent NF-KB Signaling Promotes Small Cell Lung Cancer | BioRxiv; and Xing, Y.; Lin, Y.; Zhang, Y.; Hu, J.; Liu, J.; Tian, Y.; Zhao, J.; Chen, W.; Han, B. Novel Cytoplasmic LncRNA IKBKBAS Promotes Lung Adenocarcinoma Metastasis by Upregulating IKKβ and Consequential Activation of NF-KB Signaling Pathway. Cell Death Dis 2021, 12, 1004, which is incorporated herein by reference in its entirety). Although TrxR, which regulates the cellular redox balance, including ROS levels, is a target of several gold complexes, its enzymatic activity was not modified by complex 3 treatment (FIG. 7C). The results show that complex 3 exerts its cytotoxic effects by inhibiting the activity of the proteasome and NF-kB.

### Example 16: Effects of Complex 3 on the Expression of Lung Cancer Stem Cell Markers

Lung-CSCs are characterized by the expression of the specific markers ALDH1, CD133, CD44, and NOTCH1 receptors and by the capability to form 3D-MCTSs (See: Prabavathy, D.; Swarnalatha, Y.; Ramadoss, N. Lung Cancer Stem Cells-Origin, Characteristics and Therapy. Stem Cell Investig 2018, 5, 6, which is incorporated herein by reference in its entirety).

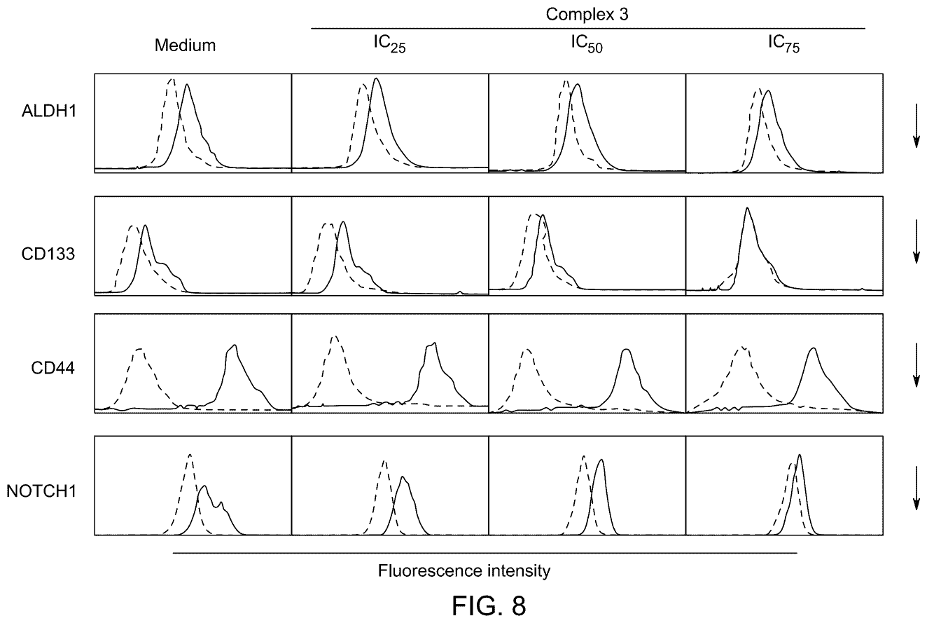

Aldehyde dehydrogenases (ALDHs) is a detoxifying enzyme that controls self-renewal, cell differentiation and chemoresistance. In lung cancer, high levels of ALDH1 correlate with poor overall survival, disease-free survival, and advanced stage of the disease and CD133 expression with therapy resistance, metastasis formation, and worse prognosis. It was observed that treatment of A549 cells with complex 3 reduced ALDH1 enzymatic of promoting activity and CD133 expression levels (FIG. 8). Complex 3 decreased CD44 (FIG. 8), an adhesion molecule involved in lung cancer cell growth, cisplatin resistance and metastasis formation, and capable to promote the expression of the immunosuppressive molecule PD-LI. Complex 3 decreased in a dose-dependent manner NOTCH1 expression (FIG. 8), whose activation regulates the expression of CSC markers (ALDH1, CD133, and CD44), the resistance to chemotherapy, and is associated with bad prognosis.

### Example 17: Comparison of the Cytotoxic Activity of Complex 3 and Cisplatin in A549-MCTSs

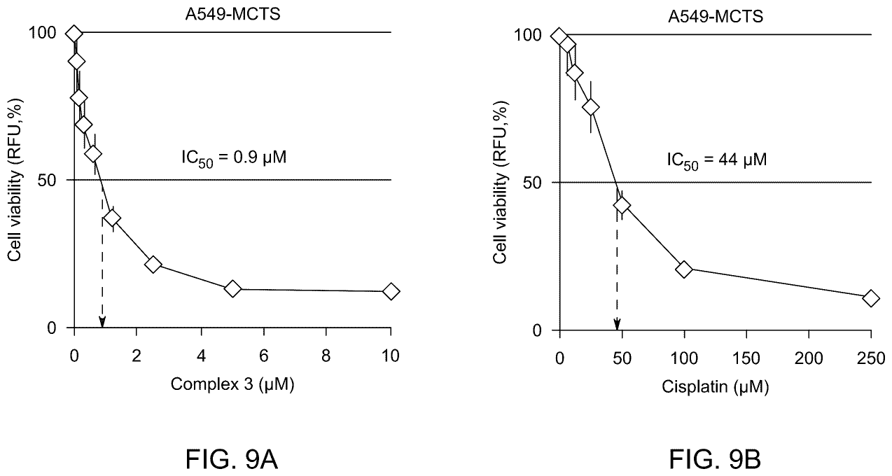

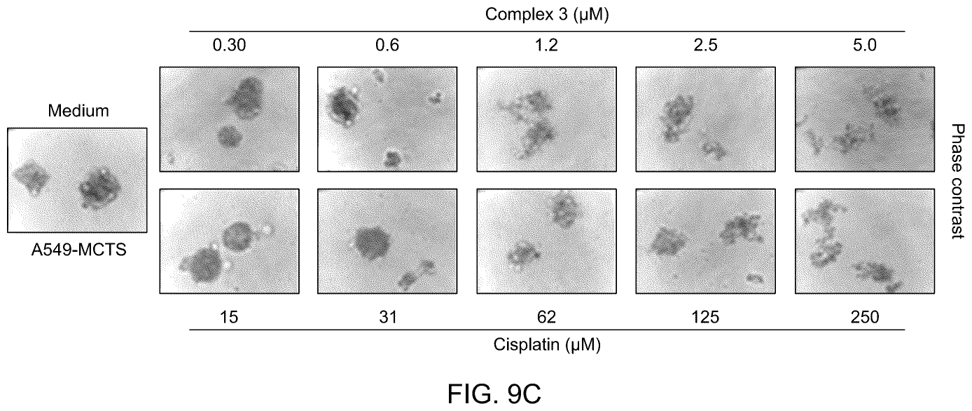

3D-MCTSs are a model for in vitro drug testing (See: Zanoni, M.; Piccinini, F.; Arienti, C.; Zamagni, A.; Santi, S.; Polico, R.; Bevilacqua, A.; Tesei, A. 3D Tumor Spheroid Models for in Vitro Therapeutic Screening: A Systematic Approach to Enhance the Biological Relevance of Data Obtained. Sci Rep 2016, 6, 19103, which is incorporated herein by reference in its entirety) and to test drug tissue penetration. Thus, the effects of complex 3 in A549-MCTSs were determined by cultivating tumor cells in non-adherent conditions on poly-HEMA coated wells. Complex 3 (0.3-10 μM) reduced the viability of A549-MCTSs in a dose-dependent manner with an IC₅₀=0.95 μM (FIG. 9A). Cisplatin (IC₅₀=44 μM), used for comparative purposes, was less active than complex 3 (FIG. 9A and FIG. 9B) with an IC₅₀ 46-fold higher than complex 3. Representative micro-photographs of A549-MCTSs treated with complex 3 (0.3-5 μM) and with cisplatin (15-250 μM) are shown in FIG. 9C.

The preparation and the spectral characterization of three new dinuclear gold(I) complexes (1-3) containing N-heterocyclic carbene and diphosphane ligands are described. The X-ray structure of complexes 2 and 3 showed that the complexes are dinuclear with a linear geometry at the gold centers. The anti-cancer properties against six cancer cell lines derived from different organs (A549 lung cancer, MCF-7 breast cancer, PC-3 prostate cancer, MG-63 osteosarcoma, A2780 and A2780cis ovarian cancer) were tested. The gold(I) complexes (1-3) exerted a cytotoxicity against all cancer cell lines tested and comparable antitumor effects against OvCa cells sensitive (A2780) and resistant to cisplatin (A2780cis). Among the synthesized complexes, complex 3 showed the highest anti-tumoral activity and was tested in lung cancer cells.

Complex 3 decreased A549 cell viability, induced apoptosis, activated caspases-3/7, increased ROS generation, and blocked cells in the G0/G1 cell cycle phase. Moreover, it decreased the 20S proteasome and NF-kB activity, but not of TrxR. Complex 3 reduced the expression of lung cancer stem cell markers (ALDH1, CD133, CD44, and NOTCH1) and was more active than the reference drug cisplatin in A549 2D cell cultures, as well as in 3D-MCTSs, due to the presence of the diphosphane ligand. These new gold (I) complexes may be used as anticancer drugs.

Numerous modifications and variations of the present disclosure are possible in light of the above teachings. It is therefore to be understood that within the scope of the appended claims, the invention may be practiced otherwise than as specifically described herein.

1: A dinuclear gold(I) complex, comprising:
two gold(I) metal ion centers;
two carbene ligands having the same structure;
a diphosphane ligand comprising two phosphine groups;
wherein the two gold metal ions are coordinated to the two phosphine groups of the diphosphine ligand in a linear geometry;
wherein each carbene ligand is independently connected to a gold metal ion centers via a carbon atom; and
wherein the dinuclear gold(I) complex has an enhanced anticancer activity compared to an equimolar amount of cisplatin administered to a subject under substantially the same conditions.

2: The dinuclear gold (I) complex of claim 1, having a formula (I) 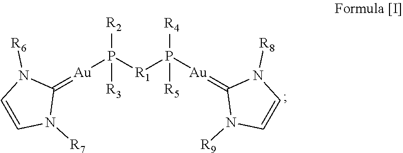
wherein R₂, R₃, R₄, R₅, R₆, R₇, R₈ and R₉ are each independently selected from the group consisting of unsubstituted or substituted 5, 6, 7 or 8-membered rings; and
wherein R₁ is independently C₁-C₆ alkyl, hydroxy-C₁-C₆ alkyl, C₁-C₆ alkyloxy, C₁-C₆ alkylthio, C₁-C₆ alkenyl, C₂-C₆ alkynyl, C₁-C₆ alkyloxy-C₁-C₆ alkyl, C₁-C₆ alkylthio-C₁-C₆ alkyl, C₁-C₆ alkylamino, di-(C₁-C₆ alkyl) amino, and C₁-C₆ alkylamino-C₁-C₆ alkyl, and wherein each of the carbon-containing radicals is optionally substituted with one or more halogen atoms.

3: The dinuclear gold(I) complex of claim 2, wherein R₆, R₇, R₈ and R₉ are each independently phenyl that is substituted in the 2 and 6 positions with at least one of hydrogen, alkyl, or aryl.

4: The dinuclear gold(I) complex of claim 3, wherein R₆, R₇, R₈ and R₉ are phenyl substituted at the 2 and 6 positions with isopropyl.

5: The dinuclear gold(I) complex of claim 2, wherein R₂, R₃, R₄, and R₅ are each independently selected from the group consisting of unsubstituted phenyl and unsubstituted cyclohexyl.

6: The dinuclear gold(I) complex of claim 2, wherein R₁ is independently selected from the group consisting of C₁-C₆ alkyl, and C₁-C₆ alkylamino-C₁-C₆ alkyl.

7: The dinuclear gold(I) complex of claim 6, wherein R₁ is independently selected from the group consisting of —C₂H₄—, —C₃H₆—, and —(C₂H₄)₂N—.

8: The dinuclear gold(I) complex of claim 1, wherein the diphosphane ligand is bis(1,2-diphenylphosphano)ethane (Dppe), and wherein the complex has a formula (II)

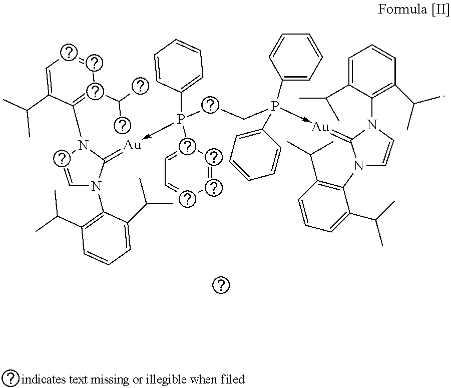

9: The dinuclear gold(I) complex of claim 8, exhibiting a cancer inhibition activity with a half maximal inhibitory concentration (IC₅₀) of 0.16 to 0.40 micromolar (μM).

10: The dinuclear gold(I) complex of claim 1, wherein the diphosphane ligand is bis(1,3-diphenylphosphano)propane (Dppp), and wherein the complex has a formula (III)

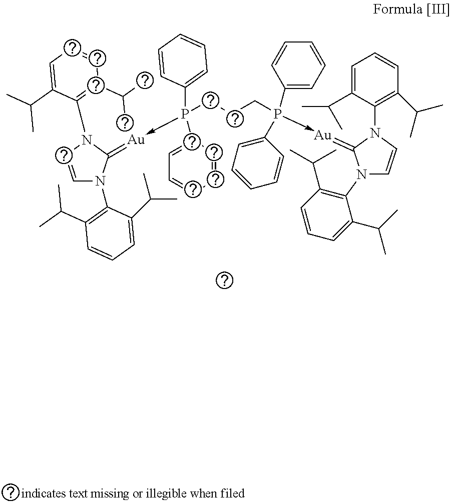

11: The dinuclear gold(I) complex of claim 10, exhibiting a cancer inhibition activity with a half maximal inhibitory concentration (IC₅₀) of 0.2 to 0.50 μM.

12: The dinuclear gold(I) complex of claim 1, wherein the diphosphane ligand is bis[2-(dicyclohexylphosphano)ethyl]amine (DCyPA), and wherein the complex has a formula

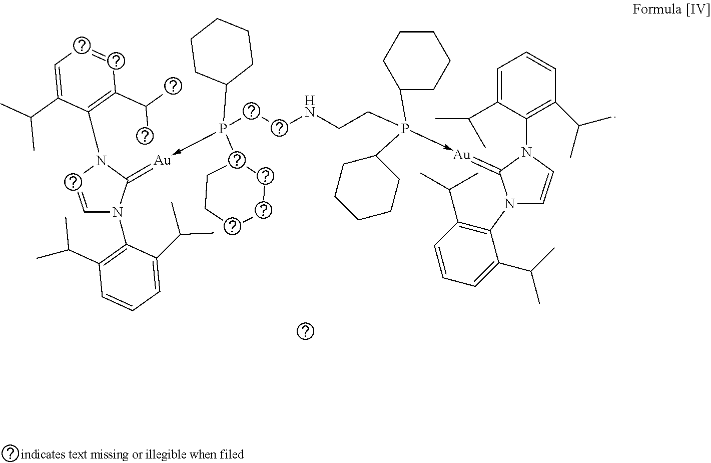

13: The dinuclear gold(I) complex of claim 12, exhibiting a cancer inhibition activity with a half maximal inhibitory concentration (IC₅₀) of 0.1 to 0.50 μM.

14: A pharmaceutical composition, comprising;
at least one dinuclear gold(I) complex of claim 1;
at least one pharmaceutically acceptable carrier or excipient; and
optionally, at least one other anticancer drug, chemotherapeutic agent, or immunopotentiator.

15: A method for inhibiting proliferation of cancer cells, comprising:
contacting the cancer cells with a cytotoxic effective amount of the dinuclear gold(I) complex of claim 1.

16: The method of claim 15, wherein the cancer cells comprise one or more cancer stem cells selected from the group consisting of a breast cancer stem, a lung cancer stem cell, a prostate cancer stem cell, an osteosarcoma cancer stem cell, an ovarian cancer stem cell, a colon carcinoma stem cell, and a melanoma stem cell.

17: The method of claim 15, wherein the cytotoxic effective amount of the dinuclear gold(I) complex is from 0.01 to 5 μM.

18: The method of claim 15, wherein the dinuclear gold(I) complex exhibits an IC₅₀ of from 0.1 to 0.5 μM for inhibiting the proliferation and inducing the apoptosis of lung cancer cells.

19: The method of claim 18, wherein the lung cancer cells are A549 lung cancer stem cells.

20: A method of treating lung cancer in a subject, comprising:
administering to the subject the dinuclear gold(I) complex of claim 1 in an amount effective to decrease the average cancer cell viability of the lung cancer by more than 10%.
